SEQUENCE LISTING (3 sequences)

SEQ ID NO: 1 - DNA, 222 bp, Mus musculus
  CDS (1)..(222)
atgtcggcgcagaccgcgagcggccccacggaggaccaggtggagatcctggagtacaacttcaacaaggtcaacaagcacccggaccccaccacgctgtgcctcatcgcagccgaggcgggtctcacggaggagcagacgcagaaatggtttaagcagcgcctggcagagtggcggcggtcagaaggcttgccttcggaatgcagatctgttacggactag

SEQ ID NO: 2 - protein, 73 aa, Mus musculus
MSAQTASGPTEDQVEILEYNFNKVNKHPDPTTLCLIAAEAGLTEEQTQKWFKQRLAEWRRSEGLPSECRSVTD

SEQ ID NO: 3 - protein, 72 aa, Homo sapiens
MSAETASGPTEDQVEILEYNFNKVDKHPDSTTLCLIAAEAGLEEETQKWFKQRLAKWRRSEGLPSECRSVTD

(19) United States
(12) Patent Application Publication
(10) Pub. No.: US 2004/0029158 A1
(43) Pub. Date: Feb. 12, 2004

(54) HOP - a novel cardiac-restricted transcriptional factor potentially useful for cardiac regeneration and specification

(76) Inventors: Eric Olson, Dallas, TX (US); Zhi-Ping Liu, Dallas, TX (US); Robert Passier, Driebergen-Rijsenburg (NL)

Correspondence Address: Steven L. Highlander, FULBRIGHT & JAWORSKI L.L.P., Suite 2400, 600 Congress Avenue, Austin, TX, 78701, US

(21) Appl. No.: 10/440,024

(22) Filed: May 16, 2003

Related U.S. Application Data
(60) Provisional application No. 60/381,221, filed on May 17, 2002.

(51) Int. Cl. C12Q 1/68; C07H 21/04; C12N 9/22; C12P 21/02; C12N 5/06; C07K 16/40

(52) U.S. Cl. 435/6; 435/69.1; 435/199; 435/320.1; 435/325; 530/388.26; 530/358; 536/23.2

(57) ABSTRACT

The present invention relates to HOP, a cardiac homeodomain protein that regulates cell differentiation and cell proliferation in cardiac, liver, lung and neuronal cells. Also disclosed are methods of using the gene and protein to treat and diagnose cardiovascular and neuronal diseases, as well as in screening methods.

The present invention claims benefit of priority to U.S. Provisional Serial No. 60/381,221, filed May 17, 2002, the entire contents of which are incorporated by reference.

### BACKGROUND OF THE INVENTION

1. Field of the Invention

The present invention relates generally to the fields of developmental biology and molecular biology. More particularly, it concerns a novel homeodomain only protein (“HOP”) that acts as a negative regulator of cell proliferation.

2. Description of Related Art

a. Heart Disease

The leading cause of morbidity and mortality in industrialized countries is heart disease, particularly heart disease that is associated with myocardial infarction. Myocardial infarction results in the loss of cardiomyocytes. Cardiomyocytes are post-mitotic cells and generally do not regenerate after birth. Furthermore, it has been discovered that they respond to mitotic signals by cell hypertrophy (Kodama et al., 1997; Pan et al, 1997) rather than by cell hyperplasia. The loss of cardiomyocytes leads to regional contractile dysfunction. In addition, the necrotized cardiomyocytes in the infarcted regions in the ventricular tissues are progressively replaced by fibroblasts to form scar tissue.

Recently, fetal cardiomyocytes transplanted in heart scar tissue limited scar expansion and prevented postinfarction heart failure (Leor et al., 1996). Although the transplantation of fetal cardiomyocytes is a proposed treatment of heart failure, it remains impractical in the clinical setting, in part because of the difficulty of obtaining fetal heart donor tissue.

Although it is known that the loss of post-mitotic cardiomyocytes results in increased morbidity and mortality, very little is known about the genes that are involved in heart development. It is known that transcription factors such as d-HAND, e-HAND (Srivastava et al., 1995), MEF-2C (Edmondson et al. 1994; Lin et al. 1997), Nkx2.5/Csx, GATA4, and TEF-1 play important roles in cardiac development (Harvey, 1996), but the lack of a model for cardiomyocyte differentiation has hindered the understanding of the interactions of these genes.

A recent report revealed that murine marrow stromal cells that are treated with 5-azacyidine, a cytosine analog capable of altering expression of certain genes that may regulate differentiation, results in a cell line that differentiates into cardiomyocytes in vitro (Makino et al., 1999). This cardiomyogenic cell line demonstrated several phenotypic characteristics that are specific to cardiomyocytes, e.g., adjoining cells via intercalated discs, forming myotubes, and beating spontaneously. In addition, the expression of cardiomyocyte specific genes, such as homeobox gene Nkx2.5, alpha-myosin heavy chain and atrial natriuretic factor, also are considered characteristic.

Although the proposed transplantation of fetal cardiomyocytes and cardiomyogenic cell lines are possible treatments, it is preferable to discover a treatment that eliminates any donor/species problems.

Another form of heart disease is cardiac hypertrophy, an adaptive response of the heart to virtually all forms of cardiac disease, including those arising from hypertension, mechanical load, myocardial infarction, cardiac arrythmias, endocrine disorders and genetic mutations in cardiac contractile protein genes. While the hypertroplic response is initially a compensatory mechanism that augments cardiac output, sustained hypertrophy can lead to dilated cardiomyopathy, heart failure, and sudden death. In the United States alone, approximately half a million individuals are diagnosed with heart failure each year, with a mortality rate approaching 50%. Because cardiac hypertrophy can be viewed as an aberration in heart growth and development, a relevant inquiry may be made into the molecular basis of cardiac tissue specification and differentiation.

Despite the diverse stimuli that lead to cardiac hypertrophy, there is a prototypical molecular response of cardiomyocytes to hypertrophic signals that involves an increase in cell size and protein synthesis, enhanced sarcomeric organization, upregulation of fetal cardiac genes, and induction of genes such as c-fos and c-myc (U.S. Pat. No. 6,372,957). The causes and effects of cardiac hypertrophy have been documented extensively, but the underlying molecular mechanisms that couple hypertrophic signals, initiated at the cell membrane to reprogram cardiomyocyte gene expression remain poorly understood. Elucidation of these mechanisms is a central issue in cardiovascular biology and is critical in the design of new strategies for prevention or treatment of cardiac hypertrophy and heart failure.

Thus, identifying new regulators of cardiomyocyte cell proliferation and differentiation is an important goal in the search for therapeutics to treat myocardial tissue damage and cardiac hypertrophy.

b. Neuronal Cell Injuries

The failure of the adult central nervous system (CNS) to regenerate after injury is a major clinical problem, affecting some 200,000 people in the United States alone. (U.S. Pat. No. 6,286,352). Despite intensive research, an effective approach in promoting significant regeneration of CNS nerve fibers remains lacking. The inability of CNS to regenerate is partly due to inhibitory factors associated with myelin, a cellular structure surrounding the nerve fibers.

There are currently few effective methods that can promote significant nerve regeneration of severed or damaged nerve fibers. A number of endogenous molecules are known to modulate neural cell growth. (U.S. Pat. No. 6,286,352). These factors may exert either attractive or repulsive action on the extension of axonal growth cones. Experiments in mammals have shown that blocking of some of the inhibitory factors by antibodies could promote regeneration of severed axons in the spinal cord and lead to functional recovery of limb movements. Thus, there remains a need to identify new regulators of neumal cell growth and regeneration.

### SUMMARY OF THE INVENTION

In one aspect, the present invention provides an isolated HOP peptide, in particular, a HOP polypeptide comprising the amino acid sequence of SEQ ID NO:2 or 3. In other aspects, the present invention provides HOP peptides comprising at least 6, 8, 10, 15, 25, 50 or more consecutive amino acids of SEQ ID NO:2 or 3. In yet other aspects of this invention, the HOP peptides described above may be fused to a heterologous amino acid sequence. In other aspects, the heterologous amino acid sequence may encode a selectable or screenable marker. In yet other aspects, the heterologous amino acid sequences comprise a nuclear localization signal.

Another aspect of the present invention provides an expression construct comprising a nucleic acid segment encoding at least 5 consecutive amino acids of SEQ ID NO:2 or 3, wherein the nucleic acid sequence is under the transcriptional control of a promoter operable in eukaryotic cells. In other aspects, the nucleic acid sequence encodes SEQ ID NO:2 or 3. In still other aspects, the promoter may be a tissue specific promoter, a constitutive promoter or an inducible promoter. The tissue specific promoter may even be active in cardiac cells or neuronal cells. In other aspects, the expression construct may comprise a non-viral vector. The non-viral vector may even be entrapped in a liposome. In other aspects, the expression construct may comprise a viral vector. The viral vector may be an adenoviral vector, an adeno-associated viral vector, a retroviral vector, a vaccinia viral vector, a herpesviral vector or a polyoma viral vector. In still other aspects, the nucleic acid segment may be positioned sense to the promoter. In other aspects, the nucleic acid segment may be positioned antisense to the promoter.

In another embodiment of the present invention there is provided an oligonucleotide consisting essentially of 12 to 200 base pairs, wherein the nucleic acid sequence comprises at least 10 consecutive bases of SEQ ID NO:1. The nucleic acid segment may even comprise at least 11, 12, 13, 14, 15, 16, 17, 18, 19 or 20 consecutive bases of SEQ ID NO:1. In other aspects, the nucleic acid segment may comprise at least 30, 40, 50, 60, 70, 80, 90, or 100 consecutive nucleotides of SEQ ID NO:1. In still other embodiments, the nucleic acid segment comprises 110, 120, 130, 140, 150, 160, 170, 180, 190 or 200 consecutive nucleotides of SEQ ID NO:1. In other aspects, the nucleic acid segment comprises SEQ ID NO:1.

In other embodiments, the present invention provides a method of inhibiting the proliferation of a cell comprising administering to the cell an effective amount of a composition that increases the level and/or activity of a HOP protein in said cell. The cell may even be a cardiac cell, a lung cell, a brain cell, a neuronal cell, or a liver cell. In other aspects, the cell may be located in a subject. In yet other aspects of this invention, the composition may comprise a HOP protein. The HOP protein may even be comprised within a liposome. In other embodiments, the composition comprises an expression construct encoding a HOP protein. The expression construct may be comprised within a viral particle. The vector may be an adenoviral vector, an adeno-associated viral vector, a retroviral vector, a vaccinia viral vector, a herpesviral vector or a polyoma viral vector. In other aspects, the expression construct may comprised within a liposome. In still other embodiments, the composition may be a small molecule.

In other aspects, the present invention provides a method of promoting the proliferation of a cell comprising administering to the cell an effective amount of a composition that reduces the levels and/or activity of a HOP protein. The cell may be a cardiac cell, a lung cell, a brain cell, a neuronal cell, or a liver cell. The cell may even be located in a subject. In still other aspects, the composition may comprise an anti-HOP antibody. The anti-HOP antibody may be comprised within a liposome. In other embodiments of this invention, the composition comprises an expression construct encoding a single chain anti-HOP antibody, a HOP ribozyme, a ds HOP RNA or a HOP antisense molecule. The expression construct may be comprised within a viral particle. The expression vector may be an adenoviral vector, an adeno-associated viral vector, a retroviral vector, a vaccinia viral vector, a herpesviral vector or a polyoma viral vector. In other aspects, the expression construct may be comprised within a liposome. In still other aspects, the composition may be a small molecule.

In another embodiment of the present invention there is provided a method of producing a HOP protein. The method comprises providing a cell comprising an expression construct comprising a nucleic acid segment encoding the sequence of SEQ ID NO:2 or 3, wherein the nucleic acid sequence is under the transcriptional control of a promoter operable in the cell and culturing the cell under conditions whereby the HOP protein is produced.

In yet another aspect of the present invention there is provided a cell comprising a nucleic acid segment sequence encoding the sequence of SEQ ID NO:2 or 3, wherein the nucleic acid segment is under the transcriptional control of a promoter, other than the native HOP promoter, that is operable in the cell. The cell may be a cardiac cell, a lung cell, a brain cell, a neuronal cell, or a liver cell. In other aspects, the promoter may be a tissue specific promoter, a constitutive promoter or an inducible promoter. The tissue specific promoter may be active in cardiac cells or neuronal cells.

In another aspect, the present invention provides a method of identifying a novel cardiac transcription factor. The method includes providing a HOP protein; contacting the HOP protein with a cellular extract; determining the binding of the HOP protein to a molecule in the cellular extract; and identifying the molecule bound to the HOP protein.

Another aspect of the present invention provides a method of diagnosing congenital heart disease in a subject. This method includes obtaining a protein-containing sample from a subject; and assessing the level of HOP protein in the sample; wherein a reduced level of HOP protein is indicative of congenital heart disease.

In still another aspect of this invention, there is provided a method of diagnosing congenital heart disease in a subject comprising. The method included obtaining a protein-containing sample from a subject; and assessing the structure of HOP protein in the sample; wherein an alteration in the structure of HOP protein is indicative of congenital heart disease.

In yet another aspect of the present, there is provided a method of diagnosing congenital heart disease in a subject. This method includes obtaining an mRNA-containing sample from a subject; and assessing the level of HOP mRNA in the sample; wherein a reduced level of HOP mRNA is indicative of congenital heart disease.

In still another aspect, the present invention provides a method of diagnosing congenital heart disease in a subject. The method includes obtaining a nucleic acid-containing sample from a subject; and identifying a sequence mutation of HOP nucleic acid in the sample; wherein a mutation in HOP nucleic acid is indicative of congenital heart disease.

In another embodiment of the present invention, there is provided a method of promoting cardiac myocyte differentiation comprising contacting an undifferentiated cardiac myocyte with a composition that increases the expression and/or activity of HOP protein in the cardiac myocyte.

In even another aspect, there is provided a method of inhibiting cardiac myocyte differentiation comprising contacting an undifferentiated cardiac myocyte with a composition that reduces the expression and/or activity of HOP protein in the cardiac myocyte.

In another embodiment, the present invention provides a method of reversing cardiac myocyte differentiation comprising contacting a differentiated cardiac myocyte with a composition that reduces the expression and/or activity of HOP protein in the cardiac myocyte.

In still another aspect of this invention, there is provided a non-human transgenic animal, cells of which lack at least one functional native HOP allele. In another embodiment, the cells of the transgenic animal may lack both functional native HOP alleles. In another aspect, the cells of the transgenic animal further comprise a HOP transgene that is under the control of regulatable promoter.

In another aspect, the present invention provides a method of treating cardiac hypertrophy in a subject comprising administering to the subject an effective amount of a composition that increases the level and/or activity of a HOP protein in said cell.

In still another aspect, the present invention provides a method of shutting off a fetal gene program in a cardiac myocyte comprising contacting the cardiac myocyte with an effective amount of a composition that increases the level and/or activity of a HOP protein in said cell.

In even another aspect of the present invention, there is provided a method of inducing neuronal tissue growth in a subject comprising administering to the subject an effective amount of a composition that reduces the levels and/or activity of a HOP protein. In other aspects, the subject has suffered a neuronal tissue injury.

In another embodiment of the present invention, there is provided an isolated and purified polynucleotide encoding the sequence of SEQ ID NO:2 or 3, or a fragment thereof. In another aspect, the polynucleotide sequence is SEQ ID NO:1.

### BRIEF DESCRIPTION OF THE DRAWINGS

The drawings accompanying and forming part of this specification are included to depict certain aspects of the invention. A clearer conception of the invention, and of the components and operation of systems provided with the invention, will become more readily apparent by referring to the exemplary, and therefore nonlimiting, embodiments illustrated in the drawings, wherein like reference numerals (if they occur in more than one view) designate the same elements. The invention may be better understood by reference to one or more of these drawings in combination with the description presented herein. It should be noted that the features illustrated in the drawings are not necessarily drawn to scale.

FIG. 1A and FIG. 1B: Sequence comparison of HOP and other homeodomain proteins: FIG. 1A. Alignment of the deduced amino acid sequences of mouse and human HOP proteins. The mouse and rat HOP sequences are identical. FIG. 1B. Sequence comparison of the homeodomain of mouse HOP with other homeodomains. Amino acid positions within the 60-amino acid homeodomain are shown. Residues that are highly conserved across the homeodomain superfamily are indicated by dots at the bottom and residue 50, which specifies DNA binding site preferences is indicated with an asterisk.

FIG. 2A and FIG. 2B: Hop expression in different human tissues and cancer cell lines. Human Hop cDNA probe was hybridized to a Clontech Human Multiple Tissue Expression Array (FIG. 2A). The corresponding tissues and cell lines are listed in FIG. 2B.

### DESCRIPTION OF ILLUSTRATIVE EMBODIMENTS

As discussed above, heart disease and its manifestations, including coronary artery disease, myocardial infarction, congestive heart failure and cardiac hypertrophy, is a major health risk in the United States today. The cost to diagnose, treat and support patients suffering from these diseases is well into the billions of dollars. Two particularly severe manifestations of heart disease are myocardial infarction and cardiac hypertrophy.

With respect to myocardial infarction, typically an acute thrombotic coronary occlusion occurs in a coronary artery as a result of atherosclerosis and causes myocardial cell death. Because cardiomyocytes, the heart muscle cells, are terminally differentiated and generally incapable of cell division, they are generally replaced by scar tissue when they die during the course of an acute myocardial infarction. Scar tissue is not contractile, fails to contribute to cardiac function, and often plays a detrimental role in heart function by expanding during cardiac contraction, or by increasing the size and effective radius of the ventricle, for example, becoming hypertrophic.

With respect to cardiac hypertrophy, one theory regards this as a disease that resembles aberrant development and, as such, raises the question of whether developmental signals in the heart can contribute to hypertrophic disease.

Similar to cardiomyocytes, neuronal cells are incapable of cell division. As noted above, the failure of the adult central nervous system (CNS) to regenerate after injury is a major clinical problem, affecting some 200,000 people in the United States alone.

The inventors have discovered a novel gene that encodes HOP, a homeodomain only protein. As disclosed herein, it is shown that HOP plays an important role in the regulation of cardiac myocyte cell differentiation and cell proliferation. The inventors have also discovered that HOP is also expressed in neuronal, liver, brain, and lung cells.

In light of HOP's role in the regulation of cell differentiation and cell proliferation, the inventors contemplate new and useful methods for treating cardiac diseases such as coronary artery disease, myocardial infarction, congestive heart failure and cardiac hypertrophy by regulating HOP expression and/or activity. In other embodiments of the present invention, the inventors contemplate new and useful methods for treating neuronal disorders and injuries by regulating HOP expression and/or activity in the neuronal cell. These and other aspects of the invention are described in greater detail below.

I. Homeodomain Proteins

Organ formation during embryogenesis requires the commitment of multipotent progenitor cells to specific cell lineages, the selective activation of tissue-specific genes, and the precise spatial organization of specialized cell types. A tightly regulated balance between cell proliferation and differentiation is also required to generate and maintain the size and shape of the mature organ. It has become apparent in recent years that these processes are controlled by combinatorial interactions between cell-specific and broadly expressed transcription factors that act through positive and negative mechanisms to govern arrays of downstream target genes in precise temporospatial patterns.

Members of the homeodomain family of transcription factors play key roles in organogenesis and embryonic patterning by interpreting positional information in the embryo and linking extracellular signals to tissue-specific gene regulatory programs. The homeodomain is an ancient DNA binding domain characterized by a helix-turn-helix motif, encoded by a DNA sequence referred to as the homeobox. Homeodomain proteins are found in all eucaryotic organisms, and over 160 potential homeobox genes have been identified in the human genome. Homeodomain proteins can be categorized into different classes based on amino acid sequence homologies within the homeodomain and on their expression patterns. A subset of homeobox genes, which confer segmental identity and positional information along the antero-posterior (AP) axis of the embryo, is located in clusters in the genome; the orientation of these genes along the chromosome correlates with their expression pattern along the AP axis. Numerous other homeobox genes are distributed throughout the genome and show highly restricted expression patterns during development.

The homeodomain is highly conserved and is comprised of 60-amino acids that adopt three α-helices, with a characteristic helix-turn-helix motif. Helix-3, referred to as the recognition helix, lies in the major groove of the DNA binding site and makes direct contact with the phosphate backbone of the DNA. Amino acid variations within the recognition helix dictate the DNA binding site specificity of different homeodomain proteins. Additional specificity of DNA binding is achieved by a flexible region, called the N-terminal arm, that extends from the first helix and wraps around the DNA to make contacts with the minor groove.

Because different homeodomain proteins can bind the same DNA sequence, target gene specificity is achieved through their association with positive and negative cofactors. Such interactions can occur between homeodomain proteins and other transcriptional regulators that bind adjacent sequences in regulatory DNA or by direct protein-protein interactions in the absence of DNA binding. A classic example of such combinatorial interactions is illustrated by the yeast homeodomain protein MATα2, which interacts with the MADS box transcription factor MCM1, resulting in repression of a-specific genes. Similarly, homeodomains of the Paired-type associate with serum response factor (SRF), a MADS box factor that controls the expression of genes involved in cell proliferation and myogenesis. Association of SRF with the phox/prxl homeodomain protein enhances the binding of SRF to DNA through a mechanism independent of homeodomain DNA binding. The association of MADS box proteins like MCM1 and SRF with homeodomain proteins couples cell identity with signal responsiveness, thereby providing a mechanism for cell type-specific responses to “generic” signals.

The cardiac-restricted homeodomain protein, Nkx2.5 has also been shown to associate with SRF, resulting in cooperative activation of cardiac gene expression. Nkc2.5, which is among the earliest markers of heart formation in vertebrate embryos is an ortholog of tinman, a homeobox gene required for formation of the Drosophila dorsal vessel. During embryogenesis, Nkx2.5 expression is initiated in cardiac precursor cells concomitant with specification of the cardiac lineage, and expression is maintained throughout the heart until adulthood. Mice homozygous for an Nkx2.5 null allele die during embryogenesis from cardiac abnormalities that include defects in looping of the heart tube, the apparent absence of a left ventricular region, and down-regulation of a subset of cardiac genes.

II. Hop

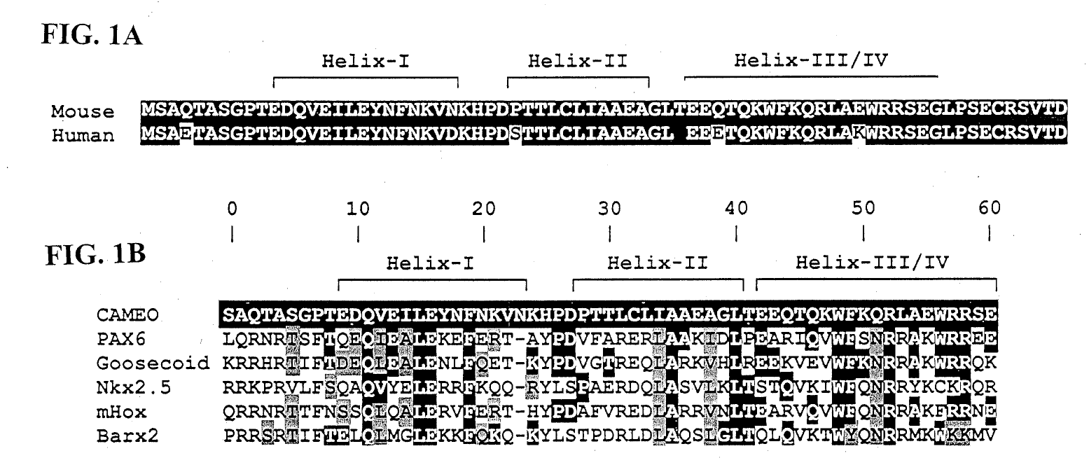

As discussed above, the inventors have discovered a novel homeobox gene that encodes a homeodomain protein they have termed homeodomain only protein (HOP) because of its prominent expression in the developing heart and its brief appearance and dramatic role in cardiogenesis. The gene encoding HOP is expressed in the developing heart and is regulated by Nkx2.5. HOP protein is an unusually small 73 amino acid homeodomain protein in mouse and rat (SEQ ID NO:2), and a 72 amino acid protein in human (SEQ ID NO:3). Mouse and rat cDNA sequences encoding identical HOP proteins and a human sequence with six amino acid differences were identified (FIG. 1A).

HOP is unusual because it is comprised simply of a homeodomain, which begins at amino acid 2. There were multiple in-frame termination codons upstream of the initiating methionine in the cDNA sequences, indicating that this is the complete coding region. Secondary structural predictions indicate that the deduced amino acid sequence of HOP would adopt three α-helices with a helix-turn-helix motif characteristic of the homeodomain.

HOP shows highest homology to Pax-6, a paired-type homeodomain protein involved in eye development, and goosecoid (gsc), which is involved in specification of anterior structures in vertebrate embryos. The HOP homeodomain contains numerous residues that are highly conserved throughout the homeodomain superfamily (FIG. 1B). For example, the WF motif at residues 48 and 49 of the homeodomain is conserved in all homeodomain proteins and partially defines this class of transcription factors. Residue 50 within the recognition helix controls the specificity of DNA binding by determining base preferences and is a signature residue for different types of homeodomains. The lysine at this position in HOP is characteristic of the paired-type homeodomain subclass. Glutamine-12, leucine-16, phenylalanine-20, alanine-30, leucine-35 and arginines-52 and -57, are also highly conserved in other homeodomains and are present in HOP.

There are also several divergent amino acids within the HOP homeodomain, some of which would be predicted to be incompatible with efficient DNA binding. For example, the nine amino acids immediately preceding helix-1 of other homeodomain proteins contain highly conserved basic residues at positions 3 and 5 that extend across the DNA binding site and make contact with the minor groove of the DNA. There are no basic residues in this region of HOP and there is a Gly-Pro sequence that would be predicted to alter the structure of this amino-terminal arm. Mutation or deletion of the basic residues in the amino-terminal arms of other homeodomain proteins severely impairs or abolishes DNA binding. In the majority of homeodomain proteins, helices 1 and 2 are separated by 5 residues. The amino acid insertion in this region of HOP would not be predicted to affect the overall conformation of the homeodomain. HOP contains a glutamine at position-51, whereas every other metazoan homeodomain contains an asparagine at this position. Residues 53 and 55 are also almost always arginine and lysine, respectively, in other homeodomains, whereas in HOP these residues are leucine and glutamic acid. Since these residues of other homeodomains make direct contact with the DNA through electrostatic interaction, the presence of hydrophobic and negatively charged residues at these positions would be unfavorable for DNA binding.

HOP is an unusual homeodomain protein that influences cardiac morphogenesis and cardiomyocyte proliferation. The fact that HOP disrupts synergistic interactions between SRF and Nkx2.5 suggests that at least some of the abnormalities observed in HOP mutant mice reflect the altered activity of these transcription factors in the absence of the negative modulatory activity of HOP. HOP can be detected in the nucleus and the cytoplasm. The protein does not contain a recognizable nuclear localization, but given its small size it should be capable of passively entering the nucleus. The properties of HOP are reminiscent of those of I-POU, a member of the POU-homeodomain family that lacks two basic residues in the N-terminal region of the homeodomain and cannot bind DNA. I-POU inhibits the activity of other POU-domain transcription factors by forming inactive heterodimeric complexes. This type of inhibitory activity is also analogous to that of the helix-loop-helix (HLH) protein, which lacks a DNA binding domain and dimerizes with basic-HLH proteins interfering with their activity. In light of the ability of homeodomain proteins to associate with GATA and bHLH transcription factors, which play key roles in cardiac development, it will be of interest to investigate whether HOP has additional positive or negative partners in the cardiac lineage.

Heart formation involves a complex series of morphogenetic events beginning with the commitment of mesodermal precursors in the cardiac crescent to a cardiac cell fate in response to cues from surrounding tissues. These cells then converge along the ventral midline of the vertebrate embryo to form a linear heart tube. Subsequent events of looping morphogenesis, chamber maturation, colonization by neural crest cells, and linkage to the vasculature gives rise to the mature multi-chambered heart. Nkx2.5 is expressed throughout the cardiac crescent rom the onset of cardiogenic specification. The severe reduction in HOP expression in Nkx2.5 mutant embryos suggests that Nkx2.5 acts upstream of HOP. HOP is also expressed in the lung, liver, neural tube and brain.

The process of cardiac development is exquisitely sensitive to the level of Nkx2.5 expression and activity. Haploinsufficiency of Nkx2.5 expression in mice and humans results in structural abnormalities in the heart that include atrial-septal, ventricular septal, and outflow tract defects, as well as atrioventricular conduction defects. Mutations have been identified in humans that alter the DNA binding, transcriptional activity, and protein-protein interactions of Nkx2.5.

Conversely, overexpression of Nkx2.5 in transgenic mice results in embryonic lethality due to abnormal cardiac morphogenesis. Overexpression of Nkx2.5 in frog and zebrafish embryos also results in expansion of the cardiac field with resulting enlargement of the heart. These findings suggest that mechanisms must exist to precisely govern Nkx2.5 activity during pre- and postnatal development.

Nkx2.5 has been shown to associate with SRF, with resulting synergistic activation of certain target genes, such as the genes encoding atrial natriuretic factor (ANF) and alpha-cardiac actin. Since Nkx2.5 is thought to act at the top of a hierarchy of cardiogenic genes, there is great interest in identifying its downstream target genes in the cardiac developmental pathway.

Cardiomyocytes lose their proliferative capacity soon after birth, and the postnatal heart grows by cellular hypertrophy rather than by cell division. The mechanism underlying cardiomyocyte cell cycle withdrawal continues to be elucidated. In HOP-deficient mice, cardiac myocytes undergo an extended period of proliferation, suggesting that HOP influences the timing of cell cycle exit of cardiomyocytes. Consistent with this notion, overexpression of HOP in HeLa cells by adenovirus infection significantly inhibits cellular proliferation as assayed by Brdu labeling and there is significant increase in G2/M cell population as assayed by FACS analysis. It is noteworthy that HOP is expressed in the trabecular region of the developing myocardium where the proliferative rate of cardiomyocyte is diminished relative to the adjacent compact zone.

Although proliferative activity of cardiac myocytes is enhanced in the absence of HOP, cardiomyocytes ultimately stop dividing in HOP mutant mice. Thus, the expression of HOP alone, is insufficient to account for the irreversible withdrawal of cardiomyocytes from the cell cycle after birth. Many of the cardiac fetal markers are up-regulated in neonatal hearts of mutant hearts despite the fact that there was no sign of hypertrophy or heart failure, suggesting that lack of HOP in mutant mice may delay the maturation of cardiomyocyte.

The mechanism underlying the over proliferation of cardiomyocytes in the neonatal hearts of HOP mutant mice is of interest. In one aspect, up-regulation of SRF activity in these mutant mice hearts may account for the phenotype, perhaps because SRF controls many “immediate-early” genes whose transcription is induced by extracellular mitogenic signals. SRF is required for phosphatidylinisitol-3-kinase (PI3K) regulated cell proliferation (Poser et al., 2000).

A variety of homeodomain proteins have been shown to associate with SRF, but HOP is unique in its ability to antagonize SRF DNA binding. The paired-type homeodomain protein phox enhances SRF activity by increasing the on-rate for DNA binding. Similarly, Nkx2.5 binds DNA cooperatively with SRF, resulting in synergistic activation of SRF target genes. Likewise, the Barx2 associates with SRF and stimulates transcription of SRF-dependent smooth muscle gene promoters. Homologies among these SRF-interacting homeodomains is shown in FIG. 1B.

III. HOP Peptides and Polypeptides

HOP is a designation assigned by the present inventors as cardiac homeodomain, because of its prominent expression in the developing heart, and because of its brief appearance and role in cardiogenesis. In addition to an entire HOP molecule, the present invention also relates to fragments of the polypeptides that may or may not retain the various functions described below. Fragments, including the N-terminus of the molecule may be generated by genetic engineering of translation stop sites within the coding region (discussed below). Alternatively, treatment of HOP with proteolytic enzymes, known as proteases, can produce a variety of N-terminal, C-terminal and internal fragments. Examples of fragments may include contiguous residues of SEQ ID NOS:2 or 3 of 6, 7, 8, 9, 10, 11, 12, 13, 14, 15, 16, 17, 18, 19, 20, 21, 22, 23, 24, 25, 30, 35, 40, 45, 50, 55, 60, 65, 70 or more amino acids in length. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

A. Variants of HOP

Amino acid sequence variants of the polypeptide can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity, as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5 ±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure (Johnson et al., 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of HOP, but with altered and even improved characteristics.

B. Domain Switching

Domain switching involves the generation of chimeric molecules using different but, in this case, related polypeptides. These molecules may have additional value in that these “chimeras” can be distinguished from natural molecules, while possibly providing the same function. For example, Upflp, Senlp, DNA helicase Hcslp, and murine Smubp-2 all provide suitable candidates for domain switching experiments.

C. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

D. Purification of Proteins

It will be desirable to purify HOP or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

E. Synthetic Peptides

The present invention also describes smaller HOP-related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979). Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

F. Antigen Compositions

The present invention also provides for the use of HOP proteins or peptides as antigens for the immunization of animals relating to the production of antibodies. It is envisioned that HOP, or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyannin (KLH) or bovine serum albumin (BSA).

IV. Nucleic Acids

The present invention also provides, in another embodiment, genes encoding HOP. See, for example, SEQ ID NO: 1, derived from mouse. The present invention is not limited in scope to these genes, however, as one of ordinary skill in the could, using these nucleic acids, readily identify related homologs in these and various other species (e.g., rat, rabbit, dog, monkey, gibbon, human, chimp, ape, baboon, cow, pig, horse, sheep, cat and other species).

In addition, it should be clear that the present invention is not limited to the specific nucleic acids disclosed herein. As discussed below, a “HOP gene” may contain a variety of different bases and yet still produce a corresponding polypeptide that is functionally, and in some cases, structurally indistinguishable, from the genes disclosed herein.

Similarly, any reference to a nucleic acid may be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate or enhance the activity of HOP.

A. Nucleic Acids Encoding HOP and Peptides Thereof

Nucleic acids according to the present invention may encode an entire HOP gene, including regulator sequences, the HOP open reading frame, a domain of HOP (e.g., sucha s those identified in FIG. 1B), or any other fragment of HOP as set forth herein. The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as a template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a given HOP from a given species may be represented by natural variants that have slightly different nucleic acid sequences but, nonetheless, encode the same protein (see Table 1 below).

As used in this application, the term “a nucleic acid encoding a HOP” refers to a nucleic acid molecule that has been isolated free of total cellular nucleic acid, including, for example, a synthetically created nucleic acid molecule. In certain embodiments, the invention concerns a nucleic acid sequence of 12 to 219 base pairs in length of SEQ ID NO:1. The term “functionally equivalent codon” is used herein to refer to codons that encode the same amino acid, such as the six codons for arginine or serine (Table 1, below), and also refers to codons that encode biologically equivalent amino acids, as discussed in the following pages.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | B | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of SEQ ID NO:1 are contemplated. Sequences that are essentially the same as those set forth in SEQ ID NO:1 may also be functionally defined as sequences that are capable of hybridizing to a nucleic acid segment containing the complement of SEQ ID NO: 1 under standard conditions.

The DNA segments of the present invention include those encoding biologically functional equivalent HOP proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

B. Oligonucleotide Probes and Primers

Naturally, the present invention also encompasses DNA segments that are complementary, or essentially complementary, to the contemplated nucleic acid segments of the present invention or to the sequence set forth in SEQ ID NO: 1. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the contemplated nucleic acid segment of SEQ ID NO:1 under relatively stringent conditions such as those described herein. Such sequences may encode entire HOP proteins or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

One method of using probes and primers of the present invention is in the search for genes related to HOP or, more particularly, homologs of HOP from other species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single-stranded and double-stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double-stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

C. Antisense Constructs

Antisense methodology takes advantage of the fact that nucleic acids tend to pair with “complementary” sequences. By complementary, it is meant that polynucleotides are those which are capable of base-pairing according to the standard Watson-Crick complementarily rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

D. Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

E. RNAi

RNA interference (RNAi) is a form of gene silencing triggered by double-stranded RNA (dsRNA). DsRNA activates post-transcriptional gene expression surveillance mechanisms that appear to function to defend cells from virus infection and transposon activity. Fire et al. (1998); Grishok et al. (2000); Ketting et al. (1999); Lin & Avery (1999); Montgomery et al. (1998); Sharp (1999); Sharp & Zamore (2000); Tabara et al. (1999). Activation of these mechanisms targets mature, dsRNA-complementary mRNA for destruction. RNAᵢ offers major experimental advantages for study of gene function. These advantages include a very high specificity, ease of movement across cell membranes, and prolonged down-regulation of the targeted gene. Fire et al. (1998); Grishok et al. (2000); Ketting et al. (1999); Lin & Avery (1999); Montgomery et al. (1998); Sharp (1999); Sharp & Zamore (2000); Tabara et al. (1999). Moreover, dsRNA has been shown to silence genes in a wide range of systems, including plants, protozoans, fungi, C. elegans, Trypanasoma and Drosophila. Grishok et al. (2000); Sharp (1999); Sharp & Zamore (1999).

Interestingly, RNAᵢ can be passed to progeny, both through injection into the gonad or by introduction into other parts of the body (including ingestion) followed by migration to the gonad. Several principles are worth note (see Plasterk & Ketting, 2000) First, the dsRNA should be directed to an exon, although some exceptions to this rule have been shown. Second, a homology threshold (probably about 80-85% over 200 bases) is required. Most tested sequences are 500 base pairs or greater. Third, the targeted mRNA is lost after RNAᵢ. Fourth, the effect is non-stoichometric, and thus incredibly potent. In fact, it has been estimated that only a few copies of dsRNA are required to knock down >95% of targeted gene expression in a cell. Fire et al. (1998).

Although the precise mechanism of RNAᵢ is still unknown, the involvement of permanent gene modification or the disruption of transcription have been experimentally eliminated. It is now generally accepted that RNAI acts post-transcriptionally, targeting RNA transcripts for degradation. It appears that both nuclear and cytoplasmic RNA can be targeted. Bosher and Labouesse (2000).

F. Vectors for Cloning, Gene Transfer and Expression

Within certain embodiments expression vectors are employed to express a HOP polypeptide product, which can then be purified and, for example, be used to vaccinate animals to generate antisera or monoclonal antibody with which further studies may be conducted. A polypeptide product may be the full length of SEQ ID NOS:2 or 3, peptides thereof, or any other product contemplated by this invention. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

(i) Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 2 and Table 3). Additionally, any other promoter/enhancer combination (for example, as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler |
| | et al., 1987; Weinberger et al., 1984; Kiledjian |
| | et al., 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo |
| | et al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson |
| | et al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi |
| | et al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987; Glue |
| | et al., 1988 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa |
| | | et al., 1987, Karin et al., |
| | | 1987; Angel et al., 1987b; |
| | | McNeall et al., 1989 |
| MMTV (mouse | Glucocorticoids | Huang et al., 1991; Lee |
| mammary | | et al., 1981; Majors et al., |
| tumor virus) | | 1983; Chandler et al., |
| | | 1983; Lee et al., 1984; |
| | | Ponta et al., 1985; Sakai |
| | | et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I | Interferon | Blanar et al., 1989 |
| Gene H-2κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor | | |
| Thyroid | Thyroid Hormone | Chatterjee et al., 1989 |
| Stimulating | | |
| Hormone α Gene | | |

Of particular interest are muscle specific promoters, and more particularly, cardiac specific promoters. These include the myosin light chain-2 promoter (Franz et al., 1994; Kelly et al., 1995), the α actin promoter (Moss et al., 1996), the troponin 1 promoter (Bhavsar et al., 1996); the Na⁺/Ca²⁺ exchanger promoter (Barnes et al., 1997), the dystrophin promoter (Kimura et al., 1997), the creatine kinase promoter (Ritchie, M. E., 1996), the α7 integrin promoter (Ziober & Kramer, 1996), the brain natriuretic peptide promoter (LaPointe et al., 1996), the αB-crystallin/small heat shock protein promoter (Gopal-Srivastava, R., 1995), and α myosin heavy chain promoter (Yamauchi-Takihara et al., 1989) and the ANF promoter (LaPointe et al., 1988).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

(ii) Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

(iii) Multigene Constructs and IES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

(iv) Delivery of Expression Constructs

There are a number of ways in which expression constructs may be introduced into cells. In certain embodiments of the invention, a vector (also referred to herein as a gene delivery vector) is employed to deliver the expression construct. By way of illustration, in some embodiments, the vector comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene delivery vectors were DNA viruses including the papovaviruses (simian virus 40, bovine papilloma virus, and polyoma) (Ridgeway, 1988; Baichwal and Sugden, 1986). Generally, these have a relatively low capacity for foreign DNA sequences and have a restricted host spectrum. They can accommodate only up to 8 kb of foreign genetic material but can be readily introduced in a variety of cell lines and laboratory animals (Nicolas and Rubenstein, 1988; Temin, 1986). Where viral vectors are employed to deliver the gene or genes of interest, it is generally preferred that they be replication-defective, for example as known to those of skill in the art and as described further herein below.

One of the preferred methods for in vivo delivery of expression constructs involves the use of an adenovirus expression vector. “Adenovirus expression vector” is meant to include those constructs containing adenovirus sequences sufficient to (a) support packaging of the construct and (b) to express a polynucleotide that has been cloned therein. In this context, expression does not require that the gene product be synthesized.

In preferred embodiments, the expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage and are able to infect non-dividing cells such as, for example, cardiomyocytes. So far, adenoviral infection appears to be linked only to mild disease such as acute respiratory disease in humans.

Adenovirus is particularly suitable for use as a gene delivery vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is important to minimize this possibility by, for example, reducing or eliminating adnoviral sequence overlaps within the system and/or to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the E3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of such adenovirus vectors is about 7.5 kb, or about 15% of the total length of the vector. Additionally, modified adenoviral vectors are now available which have an even greater capacity to carry foreign DNA.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, a preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be selected from any of the 42 different known serotypes or subgroups A-F. Adenovirus type 5 of subgroup C is a preferred starting material for obtaining a replication-defective adenovirus vector for use in the present invention. This is, in part, because Adenovirus type 5 is a human adenovirus about which a great deal of biochemical and genetic information is known, and it has historically been used for most constructions employing adenovirus as a vector.

As stated above, a preferred adenoviral vector according to the present invention lacks an adenovirus E1 region and thus, is replication. Typically, it is most convenient to introduce the polynucleotide encoding the gene of interest at the position from which the E1-coding sequences have been removed. However, the position of insertion of the construct within the adenovirus sequences is not critical to the invention. Further, other adenoviral sequences may be deleted and/or inactivated in addition to or in lieu of the E1 region. For example, the E2 and E4 regions are both necessary for adenoviral replication and thus may be modified to render an adenovirus vector replication-defective, in which case a helper cell line or helper virus complex may employed to provide such deleted/inactivated genes in trans. The polynucleotide encoding the gene of interest may alternatively be inserted in lieu of a deleted E3 region such as in E3 replacement vectors as described by Karlsson et al. (1986), or in a deleted E4 region where a helper cell line or helper virus complements the E4 defect. Other modifications are known to those of skill in the art and are likewise contemplated herein.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include administration via intracoronary catheter into one or more coronary arteries of the heart (Hammond, et al., U.S. Pat. Nos. 5,792,453 and 6,100,242) trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Another viral vector that may be used with the present invention is a herpesviral vector. Herpes simplex virus (HSV) type I and type II contain a double-stranded, linear DNA genome of approximately 150 kb, encoding 70-80 genes. Wild type HSV are able to infect cells lytically and to establish latency in certain cell types (e.g., neurons). Similar to adenovirus, HSV also can infect a variety of cell types including muscle (Yeung et al., 1999), ear (Derby et al., 1999), eye (Kaufman et al., 1999), tumors (Howard et al., 1999), lung (Kohut et al., 1998), neuronal (Garrido et al., 1999; Lachmann and Efstathiou, 1999), liver (Kooby et al., 1999) and pancreatic islets (Rabinovitch et al., 1999).

HSV viral genes are transcribed by cellular RNA polymerase II and are temporally regulated, resulting in the transcription and subsequent synthesis of gene products in roughly three discernable phases or kinetic classes. These phases of genes are referred to as the Immediate Early (IE) or alpha genes, Early (E) or beta genes and Late (L) or gamma genes. Immediately following the arrival of the genome of a virus in the nucleus of a newly infected cell, the IE genes are transcribed. The efficient expression of these genes does not require prior viral protein synthesis. The products of IE genes are required to activate transcription and regulate the remainder of the viral genome.

For use in therapeutic gene delivery, HSV must be rendered replication-defective. Protocols for generating replication-defective HSV helper virus-free cell lines have been described (U.S. Pat. No. 5,879,934; U.S. Pat. No. 5,851,826). One IE protein, Infected Cell Polypeptide 4 (ICP4), also known as alpha 4 or Vmwl75, is absolutely required for both virus infectivity and the transition from IE to later transcription. Thus, due to its complex, multifunctional nature and central role in the regulation of HSV gene expression, ICP4 has typically been the target of HSV genetic studies.

Phenotypic studies of HSV viruses deleted of ICP4 indicate that such viruses will be potentially useful for gene transfer purposes (Krisky et al., 1998a). One property of viruses deleted for ICP4 that makes them desirable for gene transfer is that they only express the five other IE genes: ICP0, ICP6, ICP27, ICP22 and ICP47 (DeLuca et al., 1985), without the expression of viral genes encoding proteins that direct viral DNA synthesis, as well as the structural proteins of the virus. This property is desirable for minimizing possible deleterious effects on host cell metabolism or an immune response following gene transfer. Further deletion of IE genes ICP22 and ICP27, in addition to ICP4, substantially improve reduction of HSV cytotoxicity and prevented early and late viral gene expression (Krisky et al., 1998b).

The therapeutic potential of HSV in gene transfer has been demonstrated in various in vitro model systems and in vivo for diseases such as Parkinson's (Yamada et al., 1999), retinoblastoma (Hayashi et al., 1999), intracerebral and intradermal tumors (Moriuchi et al., 1998), B cell malignancies (Suzuki et al., 1998), ovarian cancer (Wang et al., 1998) and Duchenne muscular dystrophy (Huard et al., 1997).

Other viral vectors may be employed as expression constructs in the present invention. Vectors derived from viruses such as vaccinia virus (Ridgeway, 1988; Baichwal and Sugden, 1986; Coupar et al., 1988) and adeno-associated virus (AAV) (Ridgeway, 1988; Baichwal and Sugden, 1986; Hennonat and Muzycska, 1984) may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al., recently introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. In general, viral vectors accomplish delivery of the expression construct by infecting the target cells of interest. Alternatively to incorporating the expression construct into the genome of a viral vector, the expression construct may be encapsidated in the infectious viral particle.

Several non-viral gene delivery vectors for the transfer of expression constructs into mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression vector may simply consist of naked recombinant DNA or plasmids comprising the expression construct. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention, transferring of a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome, another non-viral gene delivery vector. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EP 0 273 085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

V. Screening Assays

The present invention also contemplates the screening of compounds for various abilities to interact and/or affect HOP expression or function. In certain embodiments, compounds will be those useful in inhibiting or promoting the actions of HOP in cardiac differentiation and development. In other embodiments, compounds will be those useful in inhibiting or promoting cell proliferation in cardiac cells, neuronal cells, liver cells, lung cells, and/or brain cells. In the screening assays of the present invention, the candidate substance may first be screened for basic biochemical activity—e.g., binding to HOP, helicase activity, etc.—and then tested for its ability to modulate activity or expression, at the cellular, tissue or whole animal level.

A. Assay Formats

The present invention provides methods of screening for modulators of HOP. In one embodiment, the present invention is directed to a method of:

(i) providing a HOP polypeptide;

(ii) contacting the HOP polypeptide with the candidate substance; and

(iii) determining the binding of the candidate substance to the HOP polypeptide.

In yet another embodiment, the assay looks not at binding, but at HOP expression. Such methods would comprise, for example:

(i) providing a cell that expresses HOP polypeptide;

(ii) contacting the cell with the candidate substance; and

(iii) determining the effect of the candidate substance on expression of HOP.

In still yet other embodiments, one would look at the effect of a candidate substance on the activity of HOP. This may involve looking at any of a number of cardiac, neuronal, brain, lung, and/or liver cell characteristics. A model assay is found in Tang et al. (1999).

B. Inhibitors and Activators

An inhibitor according to the present invention may be one which exerts an inhibitory effect on the expression or function/activity of HOP. By the same token, an activator according to the present invention may be one which exerts a stimulatory effect on the expression or function/activity of HOP.

C. Candidate Substances

As used herein, the term “candidate substance” refers to any molecule that may potentially modulate HOP expression or function. The candidate substance may be a protein or fragment thereof, a small molecule inhibitor, or even a nucleic acid molecule. It may prove to be the case that the most useful pharmacological compounds will be compounds that are structurally related to compounds which interact naturally with HOP. Creating and examining the action of such molecules is known as “rational drug design,” and include making predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a molecule like a HOP, and then design a molecule for its abilityt to interact with HOP. Alternatively, one could design a partially functional fragment of a HOP (binding but no activity), thereby creating a competitive inhibitor. This could be accomplished by x-ray crystallography, computer modeling or by a combination of both approaches.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other compounds that may be designed through rational drug design starting from known inhibitors of hypertrophic response.

Other suitable inhibitors include antisense molecules, ribozymes, double-stranded RNA, and antibodies (including single chain antibodies).

It will, of course, be understood that all the screening methods of the present invention are useful in themselves notwithstanding the fact that effective candidates may not be found. The invention provides methods for screening for such candidates, not solely methods of finding them.

D. In Vitro Assays

A quick, inexpensive and easy assay to run is a binding assay. Binding of a molecule to a target may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to a HOP molecule or fragment thereof is provided.

The target may be either free in solution, fixed to a support, expressed in or on the surface of a cell. Either the target or the compound may be labeled, thereby permitting determining of binding. In another embodiment, the assay may measure the inhibition of binding of a target to a natural or artificial substrate or binding partner (such as a HOP). Competitive binding assays can be performed in which one of the agents (HOP for example) is labeled. Usually, the target will be the labeled species, decreasing the chance that the labeling will interfere with the binding moiety's function. One may measure the amount of free label versus bound label to determine binding or inhibition of binding.

A technique for high throughput screening of compounds is described in WO 84/03564. Large numbers of small peptide test compounds are synthesized on a solid substrate, such as plastic pins or some other surface. The peptide test compounds are reacted with, for example, a HOP and washed. Bound polypeptide is detected by various methods.

Purified target, such as a HOP, can be coated directly onto plates for use in the aforementioned drug screening techniques. However, non-neutralizing antibodies to the polypeptide can be used to immobilize the polypeptide to a solid phase.

E. In Cyto Assays

Various cell lines that express HOP can be utilized for screening of candidate substances. For example, cells containing a HOP with engineered indicators can be used to study various functional attributes of candidate compounds. In such assays, the compound would be formulated appropriately, given its biochemical nature, and contacted with a target cell.

Depending on the assay, culture may be required. As discussed above, the cell may then be examined by virtue of a number of different physiologic assays (growth, size, Ca⁺⁺ effects). Alternatively, molecular analysis may be performed in which the function of a HOP and related pathways may be explored. This involves assays such as those for protein expression, enzyme function, substrate utilization, mRNA expression (including differential display of whole cell or polyA RNA) and others.

F. In Vivo Assays

The present invention particularly contemplates the use of various animal models. Transgenic animals may be created with constructs that permit HOP expression and activity to be controlled and monitored. The generation of these animals has been described elsewhere in this document.

Treatment of these animals with test compounds will involve the administration of the compound, in an appropriate form, to the animal. Administration will be by any route the could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply.

G. Production of Modulators

In an extension of any of the previously described screening assays, the present invention also provide for method of producing modulators of HOP function or expression. The methods comprising any of the preceding screening steps followed by an additional step of “producing the candidate substance identified as a modulator of” the screened activity.

VI. Generating Antibodies Reactive With HOP

In another aspect, the present invention contemplates an antibody that is immunoreactive with a HOP molecule of the present invention, or any portion thereof. An antibody can be a polyclonal or a monoclonal antibody. In a preferred embodiment, an antibody is a monoclonal antibody. Means for preparing and characterizing antibodies are well known in the art (see, e.g., Harlow and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the monoclonal antibodies of the present invention will find useful application in standard immunochemical procedures, such as ELISA and Western blot methods and in immunohistochemical procedures such as tissue staining, as well as in other procedures which may utilize antibodies specific to HOP-related antigen epitopes. Additionally, it is proposed that monoclonal antibodies specific to the particular HOP of different species may be utilized in other useful applications In general, both polyclonal and monoclonal antibodies against HOP may be used in a variety of embodiments. For example, they may be employed in antibody cloning protocols to obtain cDNAs or genes encoding other HOP. They may also be used in inhibition studies to analyze the effects of HOP related peptides in cells or animals. HOP antibodies will also be useful in immunolocalization studies to analyze the distribution of HOP during various cellular events, for example, to determine the cellular or tissue-specific distribution of HOP polypeptides under different points in the cell cycle. A particularly useful application of such antibodies is in purifying native or recombinant HOP, for example, using an antibody affinity column. The operation of all such immunological techniques will be known to those of skill in the art in light of the present disclosure. Antibodies of the present invention may even be used to inhibit the binding of HOP to serum response factor (SRF).

Means for preparing and characterizing antibodies are well known in the art (see, e.g., Harlow and Lane, 1988; incorporated herein by reference). More specific examples of monoclonal antibody preparation are given in the examples below.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265. Typically, this technique involves immunizing a suitable animal with a selected immunogen composition, e.g., a purified or partially purified HOP protein, polypeptide or peptide or cell expressing high levels of HOP. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 l, Sp21O-Agl4, FO, NSO/U, MPC-11, MPC11-X45-GTG 1.7 and S194/5XXO Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine.

Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and mycloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

VII. Identifying Mutations in HOP

The inventors believe that HOP plays an important role in the development of cardiac tissue and, further, in the mechanisms of heart disease. Thus, in another embodiment, there are provided methods for identifying mutations in HOP expression and function. More specifically, point mutations, deletions, insertions or regulatory pertubations relating to HOP, as well as increases or decrease in levels of expression, may be assessed using standard technologies, as described below.

A. Genetic Diagnosis

One embodiment of the instant invention comprises a method for detecting variation in the expression of HOP. This may comprise determining the level of HOP or determining specific alterations in the expressed product.

A suitable biological sample can be any tissue or fluid. Various embodiments include cells of the skin, muscle, facia, brain, prostate, breast, endometrium, lung, head & neck, pancreas, small intestine, blood cells, liver, testes, ovaries, colon, skin, stomach, esophagus, spleen, lymph node, bone marrow or kidney. Other embodiments include fluid samples such as peripheral blood, lymph fluid, ascites, serous fluid, pleural effusion, sputum, cerebrospinal fluid, lacrimal fluid, stool or urine.

Nucleic acid used is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Various types of defects may be identified by the present methods. Thus, “alterations” should be read as including deletions, insertions, point mutations and duplications. Point mutations result in stop codons, frameshift mutations or amino acid substitutions. Somatic mutations are those occurring in non-germline tissues. Germ-line tissue can occur in any tissue and are inherited. Mutations in and outside the coding region also may affect the amount of HOP produced, both by altering the transcription of the gene or in destabilizing or otherwise altering the processing of either the transcript (mRNA) or protein.

It is contemplated that other mutations in the HOP genes may be identified in accordance with the present invention. A variety of different assays are contemplated in this regard, including but not limited to, fluorescent in situ hybridization (FISH), direct DNA sequencing, PFGE analysis, Southern or Northern blotting, single-stranded conformation analysis (SSCA), RNAse protection assay, allele-specific oligonucleotide (ASO), dot blot analysis, denaturing gradient gel electrophoresis, RFLP and PCR™-SSCP.

(i) Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

In preferred embodiments, the probes or primers are labeled with radioactive species (³²P, ¹¹C, 35S, ³H, or other label), with a fluorophore (rhodamine, fluorescein) or a chemillumiscent (luciferase).

(ii) Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., (1989).

(iii) Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

(iv) Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

(v) Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al., 1989. For example, chromophore or radiolabel probes or primers identify the target during or following amplification.

One example of the foregoing is described in U.S. Pat. No. 5,279,721, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al., 1994). The present invention provides methods by which any or all of these types of analyses may be used. Using the sequences disclosed herein, oligonucleotide primers may be designed to permit the amplification of sequences throughout the HOP gene that may then be analyzed by direct sequencing.

(vi) Kit Components

All the essential materials and reagents required for detecting and sequencing HOP and variants thereof may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe.

B. Immunologic Diagnosis

Antibodies of the present invention can be used in characterizing the HOP content of healthy and diseased tissues, through techniques such as ELISAs and Western blotting. This may provide a screen for the presence or absence of cardiomyopathy or as a predictor of heart disease.

The use of antibodies of the present invention, in an ELISA assay is contemplated. For example, anti-HOP antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for HOP that differs the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° C. to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

VIII. Treatment of Diseases and Injuries by Regulating HOP Expression and Activity

As discuss above, the present invention provide methods for altering the proliferation of certain cell types by up- or down-regulating HOP expression and/or activity. In particular, given the expression of HOP in cardiac and nerve cells, the inventors contemplate the use of HOP modulators to treat such conditions as cardiac hypertrophy and spinal cord injuries. These modulators may also find use in various in vitro embodiments where they are used to alter the growth patters of cells in culture.

A. Genetic Based Therapies

One of the therapeutic embodiments contemplated by the present inventors is the intervention, at the molecular level. Specifically, the present inventors intend to provide an expression construct capable of providing HOP or a HOP stimulatory or inhibitory protein to a cell. The lengthy discussion of expression vectors and the genetic elements employed therein is incorporated into this section by reference. Particularly preferred expression vectors are viral vectors such as adenovirus, adeno-associated virus, herpesvirus, vaccinia virus and retrovirus. Also preferred are liposomally-encapsulated expression vectors.

Those of skill in the art are aware of how to apply gene delivery to in vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

B. Combined Therapy

In many clinical situations, it is advisable to use a combination of distinct therapies. Thus, it is envisioned that, in addition to the HOP-based therapies described above, one would also wish to provide to the patient more “standard” pharmaceutical therapies for cardiac hypertophy, cardiac failure, high blood pressure, arrythmia or other heart-related disorder. These include, but are not limited to, so-called “beta blockers”, anti-hypertensives, cardiotonics, anti-thrombotics, vasodilators, hormone antagonists, endothelin antagonists, calcium channel blockers, phosphodiesterase inhibitors, angiotensin type 2 antagonists and cytokine blockers/inhibitors. Also envisioned are combinations with agents identified according to the screening methods described herein.

Combinations may be achieved by contacting cardiac cells with a single composition or pharmacological formulation that includes both agents, or by contacting the cell with two distinct compositions or formulations, at the same time, wherein one composition includes the expression construct and the other includes the agent. Alternatively, HOP therapy may precede or follow the other agent treatment by intervals ranging from minutes to weeks. In embodiments where the other agent and HOP therapy are applied separately to the cell, one would generally ensure that a significant period of time did not expire between the time of each delivery, such that the agent and the HOP therapy would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a HOPt herapeutic, or the other agent will be desired. In other embodiments, Various combinations may be employed, where HOP is “A” and the other agent is “B”, as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are contemplated as well.

C. Formulations and Routes for Administration to Patients

Where clinical applications are contemplated, it will be necessary to prepare pharmaceutical compositions—proteins, antibodies, expression vectors, virus stocks and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal, intravascular or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

IX. Transgenic Animals

Transgenic animals may be useful in methods for screening for and identifying agents that modulate a function or activity of HOP, and thereby alleviate pathology related to the over or under expression of these molecules. The use of constitutively expressed HOP provides a model for over- or unregulated expression. Also, transgenic animals which are “knocked out” for HOP will find use in analysis of developmental aspects of HOP.

In a general aspect, a transgenic animal is produced by the integration of a given transgene into the genome in a manner that permits the expression of the transgene. Methods for producing transgenic animals are generally described by Wagner and Hoppe (U.S. Pat. No. 4,873,191), Brinster et al. 1985) and in “Manipulating the Mouse Embryo; A Laboratory Manual” 2nd edition (eds., Hogan, Beddington, Costantimi and Long, Cold Spring Harbor Laboratory Press, 1994).

Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mice, mammals, and fish.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris,pH 7.4, and 1 mM EDTA) and purified on an Elutip-DTM column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 μg/ml in 5 mM Tris, pH 7.4 and 0.1 mM EDTA.

Other methods for purification of DNA for microinjection are described in Hogan et al. Manipulating the Mouse Embryo (Cold Spring Harbor Laboratory, Cold Spring Harbor, N.Y., 1986), in Palmiter et al. Nature 300:611 (1982); in The Qiagenologist, Application Protocols, 3rd edition, published by Qiagen, Inc., Chatsworth, Calif.; and in Sambrook et al. Molecular Cloning: A Laboratory Manual (Cold Spring Harbor Laboratory, Cold Spring Harbor, N.Y., 1989).

In an exemplary microinjection procedure, female mice six weeks of age are induced to superovulate with a 5 IU injection (0.1 cc, ip) of pregnant mare serum gonadotropin (PMSG; Sigma) followed 48 hours later by a 5 IU injection (0.1 cc, ip) of human chorionic gonadotropin (hCG, Sigma). Females are placed with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA, Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Cloning and sequencing of mouse HOP. Public EST databases were screened using a consensus sequence for the homeodomain, combined with a text string-search, in order to identify novel heart-enriched homeodomain proteins. EST AA222563 from a 6-week old mouse heart cDNA library displayed a 39-41% homology with the homeodomain transcription factors Goosecoid and Pax-6. This 1 kb EST clone (Genome Systems) was sequenced using an automated DNA sequencer. Although the sequence revealed 5′ and 3′ stop codons, attempts were made to obtain more 5′ upsteram sequence. Using the 1 kb insert as a probe, a mouse heart cDNA library in a Uni-ZAP XR vector (Stratagene) was screened, yielding multiple positive clones. Full-length cDNA clones were also obtained by 5′ rapid amplification of cDNA ends (RACE). None of these techniques yielded additional 5′ coding sequence beyond the open reading frame shown in FIG. 1A.

The inventors also compared EST AA222563 with ESTs A1848177 and BQ109181 (Applicants incorporate EST A1848177 and EST BQ109181 herein by reference). These comparisons disclosed that EST A1848177 and EST BQ109181 contained mutations that are not present in EST AA222563.

RNA analysis. A mouse multiple tissue Northern blot (Clontech) containing 2 ug of poly A+ mRNA from adult tissues was hybridizaed with a ³²P-labeled probe prepared from EST A222563. Hybridization was performed under high stringency conditions at 65° C. for 16 hours. Following hybridization, the filter was washed in 2×SSC at room temperature, followed by washes in 0.2×SSC/0.1% SDS at 65° C. Filters were exposed to X-ray film for 18 hours and developed afterwards.

In Situ Hybridization. Mouse embryos at ages ranging from E7.5 to E16.5 were fixed overnight in 4% paraformaldehyde in phosphate-buffered saline (PBS), treated with DEPC. In situ hybridization of whole embryos or paraffin sections was performed as described previously, using sense and antisense probes prepared from EST A222563.

Preparation of HOP antibody. The complete open reading frame of HOP was subcloned into the pGEX-KG cloning vector to generate a HOP-GST fusion protein. After transformation in BL21D3 competent cells (Stratagene), IPTG (100 mM) was added and incubated for 2 hours at 37° C. for induction of protein expression. Cell lysates were prepared, and HOP-GST protein was isolated by binding to glutathione-sepharose beads (Amersham) followed by digestion with thrombin to cleave GST. Purified HOP protein was used as an antigen for the preparation of polyclonal antibodies in rabbits (Biosynthesis). Sera from rabbit were purified using protein A sepharose beads and used for Western blots and immunocytochemistry.

Immunocytochemistry and Western analysis. To determine the subcellular localization of HOP protein in rat neonatal cardiomyocytes, cells were fixed in 10% formalin, followed by 3 washes in PBS and a 30 min incubation in 0.1% NP40/BSA/PBS. Cardiomycocytes were then incubated with a 1:200 dilution of the anti-HOP antiserum in NP40/BSA/PBS for 60 min at RT. This was followed by 3 washes in PBS and 60 min incubation with the secondary goat-anti-rabbit antibodies. Cardiomyocytes were washed in PBS, finished with aquamount.

Generation of HOP mutant mice. A HOP genomic clone was isolated from a lambda FIX II mouse 129/Sv genomic library using a radioactively labeled HOP cDNA probe (NotI-EcoRI insert of EST AA222563 in pT7T3). An 11 kb genomic DNA fragment was isolated that contained two exons, encoding the predicted full length protein. To construct the targeting vector, the inventors used a plasmid vector containing nuclear LacZ (nLacZ), PolNeo and HSV-TK cassettes, as previously described. The 3′ arm comprising a 0.8 kb NotI-Xho fragment and a 5′ arm comprising a 4.5 kb KpnI-SalI fragment were cloned into the targeting vector. The complete protein coding region of protein was replaced with an nLacZ and a PolNeo cassette. SM-1 ES cells derived from a 129/SvEv mouse strain were cultured on an irradiated LIF-producing STO feeder layer, transfected with the targeting vector after linearization by digestion with NotI, and doubly selected in G418 and FIAU, as previously described. ES clones were picked and homologous recombination was confirmed by Southern analysis. Recombinant ES cell clones were injected into blastocysts obtained from C57B16/J females. Chimeras were mated with C57BL6/J females to obtain F1 mice carrying the targeted allele. Genomic DNA was prepared from tail biopsies at postnatal day 21, as previously described. For southern blot analysis, 10 ug of genomic DNA was digested with SacI or EcoRI and hybridized with a 3′ and 5′ probes representing sequences external to the targeted region.

Immunohistochemistry. Hearts were prepared for sectioning by dehydration in ethanol and embedding in paraffin. Sections were cut at 10 um intervals and dried on microscope slides. Paraffin was removed with xylene, and sections were stained with hematoxylin/eosin and covered. For immunostaining, sections were deparaffinized in xylene, hydrated through graded ethanols to PBS and permeabilized in 0.3% Triton-X 100 in PBS. Nonspecific binding was blocked by 1.5% normal goal serum in PBS and anti-phospho-histone H3 rabbit polyclonal antibody (Upstate Biotechnology) was applied at a 1:200 dilution in 0.1% BSA in PBS overnight at 4° C. Sections were washed in PBS and fluorescein-conjugated anti-rabbit antibody (Vector Laboratories) was applied at a 1:200 dilution in 1% normal goat serum for 1 hr at room temp. Nuclear staining with Hoescht 33342 was performed and sections were coverslipped with vectashield mounting media.

Quantitation of cardiomyocyte cell numbers. Hearts were dissected from mice, washed in 10% PBS, placed in 10% neutral buffered formalin, and fixed at 4° C. Hearts were then treated with 50% KOH overnight at 4° C., followed by extensive washing in distilled water. Specimens were dried and vortexed before addition of PBS containing Hoechst 33342 to yield a final suspension of 10 mg fixed tissue/ml. Dissociated cardiomyocytes were counted with a Fuchs-Rosenthal counting chamber (Hausser Scientific).

Glutathione S-transferase pull-down assays. GST-fusion of full length SRF was generated and purified, as previously described. Full-length HOP protein was in vitro-translated in rabbit reticulacyte lysate (Promega, Madison, Wis.) supplemented with [³⁵S]-methionine (Amersham Phamacia Biotech). In vitro binding experiments were performed as described previously.

Immunoprecipitation and Western blotting analysis. Expression vector containing Flag-tagged HOP was cotransfected into 293T cells with HA-tagged wild-type and mutant SRF expression plasmids. Cells were harvested 30 hrs later and lysed in 1 mL of ice-cold PBS buffer supplemented with complete protease inhibitors (Roche), 0.5% TX-100, 1 mM EDTA, and 40 units of DNAase I (Roche Molecular Biochemicals, Mannheim, Germany). The immunoprecipitations were carried out by incubating 500 ul lysate with 20 ul Flag-sepharose (Sigma, St. Louis) at 4° C. for 3 hrs. The beads were washed 2 times with lysis buffer and boiled in 1×SDS sample buffer for 5 min before electrophoresis. Immunoprecipitation products were analyzed by Western blotting using rat anti-HA antibody (Roche Molecular Biochemicals, Mannhein, Germany) following the procedure as described.

Gel mobility shift assays. SRF (0.2 ug DNA) was cotranslated in vitro in the presence of increasing amount of HOP (0.2 to 0.8 ug) in 25 ul total volume with a TNT T7-coupled reticulocyte lysate system (Promega). Gel mobility shift assays were performed with ³²P-labeled oligonucletides containing c-fos SRE as described (Chang et al., 2001).

### Example 2

### Results

Expression pattern of HOP. The expression pattern of HOP during mouse embryogenesis was determined by in situ hybridization. HOP transcripts were first detected in trophoblasts within extraembryonic membranes at E7.5 (data not shown). At E7.75, HOP transcripts were detected in the lateral wings of the cardiac crescent and in the anterior head folds. Expression of HOP in the cardiac crescent lags behind that of Nkx2.5 by about 8 hrs and is less extensive; whereas Nkx2.5 is expressed throughout the entire cardiac crescent, HOP expression is restricted to the lateral domains. At E8.0, HOP expression was detected in parallel domains along the length of the newly formed linear heart tube and in the head folds. At E8.25 to 9.5, expression was also observed in the branchial arches and lateral mesoderm dorsal to the heart. Expression was maintained throughout the ventricular and atrial chambers of the heart through E14.5. Beginning at about E12.5, we also observed HOP expression within the periventricular zone of the neural tube, and by E16.5, expression of HOP was observed in the lungs. A single HOP transcript of 1.3 kb was detected in adult mouse heart, lung, brain and liver.

Down-regulation of HOP expression in Nkx2.5 mutant mice. To begin to determine where HOP might act within the network of genes that controls heart formation, the inventors examined its expression in a series of mouse mutants with abnormalities in specific steps of cardiac development. HOP expression was dramatically downregulated in Nkx2.5 mutant embryos, which fail to form a left ventricular chamber. HOP was expressed at normal levels in mice lacking the bHLH transcription dHAND, which fail to form a right ventricle, or MEF2C, which show abnormal atrial and ventricular development (data not shown). The inventors conclude that HOP expression is dependent on Nkx2.5 during the early stages of heart development.

Genration of HOP mutant mice. To further investigate the function of HOP during mouse embryogenesis, the inventors generated HOP null mice by gene targeting. The HOP gene contains two exons separated by an intron. The targeting vector deleted exon 1, which encodes amino acids 1-48, along with the intron, and introduced a nuclear-localized lacZ protein. Mice bearing the targeted HOP allele were identified by Southern blot analysis.

Mice heterozygous for the HOP null mutation were viable and were intercrossed to obtain HOP null offspring. The inventors generated HOP mutants in the isogenic 129sv background, as well as in the mixed C57BL6 background. In both backgrounds, HOP null mice were viable and fertile. However, viable homozygous mutants were obtained at frequencies approximately 20% lower than predicted Mendelian ratios. These findings suggested that the HOP mutant phenotype resulted in embryonic lethality with variable penetrance, and that HOP was not absolutely essential for pre- or postnatal development. Northern blot analysis of RNA from heart and liver of HOP-null mice confirmed that the targeted mutation eliminated all detectable expression of HOP mRNA, as expected from the gene targeting strategy. HOP transcripts were also undetectable by RT-PCR of RNA from the hearts of adult HOP-null mice.

LacZ expression from the targeted HOP allele. Gene targeting strategy introduced an in frame nuclear lacZ cassette into HOP. LacZ staining of embryos heterozygous or homozygous for the HOP mutation showed a pattern corresponding to that of the endogenous HOP gene. However, in contrast to the endogenous gene, the inventors did not detect significant expression of the targeted lacZ gene in the heart until E9.5 (data not shown). Cardiac expression of lacZ was clearly detectable at E10.5 and was especially prominent after E11.5. LacZ staining was also observed within the neural tube, forming two parallel columns of stained cells along the AP axis of the embryo. Serial sections of the heart at E11.5 revealed the highest levels of lacZ staining within cardiomyocyte nuclei in the trabecular zone, where proliferation is diminished relative to the adjacent compact zone in the ventricular wall. This cardiac staining pattern persisted at E13.5. Within the neural tube, staining was localized mainly to the periventricular zone.

Cardiomyocyte hyperplasia in HOP mutant mice. Analysis of viable HOP mutant mice revealed enlarged hearts with increased ventricular wall thickness compared to control littermates. Cardiac enlargement was especially apparent during the first week of post-natal life, but was also seen in adults. At postal day 2 (P2), the heart weight to body weight ratio (HW/BW) in mutant mice is ca. 20% larger than wild-type littermates. This increase of HW/BW ratio in mutant mice was maintained until adulthood.

Histologic examination of mutant hearts suggested that the increase in size was largely due to an increase in cardiomyocyte cell number. This was confirmed by dissociating hearts from wild-type and mutant littermates and performing cell counts following staining with anti-actin antibody to identify cardiac myocytes. There was approximately 35% increase in the number of cardiac myocytes in mutant hearts at post-natal day 1 (P1). An increase in cardiomyocyte cell number was also observed at 1 and 6 months of age. The size of cardiomyocytes was similar in mutant and wild-type mice up to 4 weeks of age. At six months of age, a subset of mutant mice developed severe cardiac hypertrophy.

Delayed withdrawal of HOP mutant cells from the cell cycle. Cardiac muscle cells normally begin to withdraw from the cell cycle at birth. To determine whether the increase in cardiomyocyte cell number was associated with prolonged proliferation, the inventors stained histological sections of postnatal day 1 heart (P1) with anti-phospho-Histone H3 antibody, a mitosis marker. Whereas phospho-H3-positive cells were observed only occasionally in wild-type hearts, they were widespread in the mutant hearts. Quantitation of PH3-positive cells revealed a 19-fold increase in the mutant compared to wild-type. No phospho-H3 positive cardiomyocytes were detected in either mutant and wild-type littermates at 4 weeks of age, suggesting the extended cardiomyocyte proliferation only occurred during the neonatal period. The inventors confirmed that these cells were cardiac myocytes, rather than fibroblasts, by double staining with anti-desmin antibody (data not shown).

In light of the preferential lacZ expression of HOP in the trabecular region of the developing heart, in which the proliferative rate of cardiomyocytes is reduced relative to the adjacent compact zone, and increased proliferation in knockout mice of HOP, anti proliferation assay was performed to test whether HOP might suppress cell proliferation. Indeed, in Hela cells ectopically expressed HOP using an adenoviral expression vehicle, BrdU incorporation was inhibited significantly compare with lacZ control.

Altered gene expression in hearts from HOP mutant mice. To investigate the molecular basis for the aberrant proliferation of cardiac myocytes in HOP mutant mice, the inventors performed microarray and RT-PCR analysis on neonatal hearts from wild-type and mutant mice. A list of genes that were up-regulated in HOP mutant mice is shown in Table 4.

TABLE 4

| | |
|---|---|
| | Microarray |
| Genes | RT-PCR |

| | | |
|---|---|---|
| 1. Proliferation-related genes | | |
| Cyclin D3 | +3 | |
| BTEB1 | +5 | |
| Mouse growth factor-induced delayed early | +3 | |
| response protein | | |
| Homer neuronal immediate early gene | 3 | |
| 2. Cardiac hypertrophy related genes | | |
| b-MHC | 11 | 10 |
| Myosin-binding protein | 10 | |
| Sk. MLC, phosphorylatable | 3 | |
| Cardiac MLC, alkali | 3 | |
| SERCA1 | 3 | |
| ANF | | 5 |
| BNP | | 5 |
| Sk a-actin | | 2 |
| 3. Smooth muscle cell specific genes | | |
| Actinin alpha 2 | 3 | |
| Calponin 1 | 3 | |
| SM22 | | 2 |
| Sm a-actin | | 2 |

Notably, several proliferation-related genes were up-regulated in the hearts of homozygous mutants. These included genes encoding cyclin D3, BTEB1, growth factor-induced delayed early response protein, and homer, a neuronal immediate early gene. HOP mutants also showed elevated expression of fetal genes that typically associated with cardiac hypertrophy, including α-MHC, cardiac α-actin, skeletal α-actin, atrial natriuretic factor (ANF), b-type natriuretic factor (BNP), SERCA, and MLC. Interestingly, several smooth muscle-restricted genes, including those encoding actinin alpha 2, calponin, smooth muscle α-actin, and SM22 were up-regulated in mutant hearts. Sm α-actin and SM22 are known to be down-stream targets of SRF.

RT-PCR was performed to compare expression level of genes known to be involved in cardiac development. The mRNA level of BNP and bMHC was significantly upregulated in postnatal day 1 mutants compare with same age wild type. The level of ANF and alpha skeletal actin was slightly upregulated. GAPDH showed equal amount of mRNA was used for RT-PCR. Adult northern showed bMHC expression was upregulated in mutant animals. ANF level didn't seem to change between mutant and wild type animals. GAPDH showed equal amount of mRNA was used for northern blot analysis.

Association of HOP with SRF and interference with SRF-dependent transcription by HOP. In light of the ability of Nkx2.5 and other homeodomain proteins to interact with SRF, the inventors tested whether HOP could associate with SRF using GST-pull down assay. GST-SRF was expressed in E. coli, purified, and mixed with in vitro-translated [³⁵S]methionine-labeled HOP in rabbit reticulate lysate. [³⁵S]-labeled HOP was retained by GST-SRF immobilized on glutathione-sepharose beads. No [³⁵S]-labeled HOP was detected on the control GST glutathione-sepharose beads.

To demonstrate that the interaction between SRF and HOP also occurs in vivo and further map the region of SRF that interacts with HOP, 293T cells were cotransfected with Flag-tagged HOP and HA-tagged wild-type and mutant SRF constructs. Immunoprecipitation with an anti-Flag antibody and subsequent Western blotting with anti-HA antibody revealed that HOP interacts with SRF DNA-binding domain (MADS box).

The fact that HOP interacts with MADS box of SRF suggests that HOP may interfere with SRF-DNA binding. The inventors performed SRF gel mobility shift assay with ³²P-labeled oligonucleotides probe containing c-fos serum responsive element (SRE). Consistent with its inability to bind any DNA sequences, HOP failed to bind to the c-fos SRE probe. But when mixed with SRF in the in vitro-translated rabbit reticular lysate, HOP inhibits SRF binding to c-fos SRE probe in a dose-dependent manner.

To assess the significance of the interactions between SRF and HOP, a reporter plasmid containing luciferase gene under the control of cardiac a-actin promoter was cotransfected into COS-1 cells with expression plasmids.

SRF synergistically activates aCA promoter with Nkx 2.5/GATA-4 (ca. 30-fold), this synergy was abolished in a dose-dependent manner by HOP, suggesting that HOP can function as SRF repressor in SRF-dependent gene transcription.

To further investigate the mechanism of action of HOP, the inventors analyzed the subcellular distribution of the protein in transfected COS cells. The HOP protein was localized primarily to the nucleus, but weak cytoplasmic staining could also be observed. The inventors also tested whether HOP was able to bind DNA by performing gel mobility shift assays with bacterially-expressed HOP protein and a series of oligonucleotide probes representing the binding sites for other homeodomain proteins. No detectable DNA binding activity of HOP was observed in these assays. Similarly, PCR-mediated binding site selection assays using bacterially-expressed HOP and random oligonucleotide probes failed to reveal DNA binding.

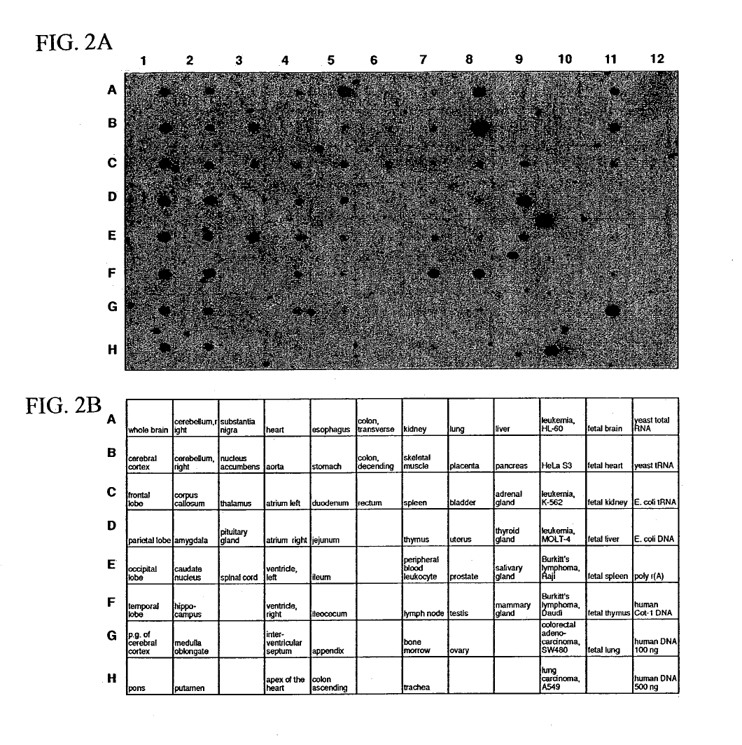

HOP expression in different human tissues and cancer cell lines. A human HOP cDNA probe was used to probe a Human Multiple Tissue Expression Array (Clontech; FIG. 2A). The corresponding tissues and cell lines are listed in FIG. 2B. HOP expression was downregulated in various cancer cells, further supporting the role of HOP in the regulation of cell growth.

HOP overexpression studies. In order to assess the impact of ectopic HOP expression on cellular proliferation, Hela cells were infected with an adenovirus expression vector comprising a human HOP cDNA (“adeno-HOP”). Cells were pulse-labeled with Brdu for 30 min, fixed, and stained with anti-Brdu antibody and Dabi. The number and stain intensity of cells stained with Brdu were significantly decreased (15%) in adeno-HOP infected cells, as compared to Hela cells infected with no or control β-gal adenovirus (25%). This result indicates that overexpression of HOP in Hela cells inhibits cell proliferation. In a similar experiment, Hela cells infected with adeno-HOP virus show increased number of cells containing multiple nuclei (36%) compared to cells infected with no virus or control P-gal virus (approx. 18%). The results of each of these experiments further demonstrate the role of HOP in cell growth and regulation.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,554,101

U.S. Pat. No. 5,354,855

U.S. Pat. No. 5,792,453

U.S. Pat. No. 6,100,242

U.S. Pat. No. 4,196,265

U.S. Pat. No. 4,683,195

U.S. Pat. No. 4,683,202

U.S. Pat. No. 4,800,159

U.S. Pat. No. 4,883,750

U.S. Pat. No. 5,279,721

U.S. Pat. No. 4,873,191

U.S. Pat. No. 6,372,957

U.S. Pat. No. 6,286,352

Angel, Bauman, Stein, Dellus, Rahmsdorf, and Herrlich, “12-O-tetradecanoyl-phorbol-13-acetate Induction of the Human Collagenase Gene is Mediated by an Inducible Enhancer Element Located in the 5′Flanking Region,” Mol. Cell. Biol., 7:2256, 1987a.

Angel, Imagawa, Chiu, Stein, Imbra, Rahmsdorf, Jonat, Herrlich, and Karin, “Phorbol Ester-Inducible Genes Contain a Common cis Element Recognized by a TPA-Modulated Trans-acting Factor,” Cell, 49:729, 1987b

Atchison and Perry, “Tandem Kappa Immunoglobulin Promoters are Equally Active in the Presence of the Kappa Enhancer: Implications for Model of Enhancer Function,” Cell, 46:253, 1986.

Atchison and Perry, “The Role of the Kappa Enhancer and its Binding Factor NF-kappa B in the Developmental Regulation of Kappa Gene Transcription,” Cell, 48:121, 1987.

Baichwal and Sugden, “Vectors for gene transfer derived from animal DNA viruses: Transient and stable expression of transferred genes”, In: Gene Transfer, Kucherlapati R, ed., New York, Plenum Press, pp. 117-148, 1986.

Banerji, Olson, and Schaffner, “A lymphocyte-specific cellular enhancer is located downstream of the joining region in immunoglobulin heavy-chain genes,” Cell, 35:729, 1983.

Barany and Merrifield, The Peptides, Gross and Meienhofer, eds., Academic Press, New York, pp. 1-284, 1979.

Barnes, Cheng, Dawson, Menick, “Cloning of cardiac, kidney, and brain promoters of the feline ncxl gene,” J. Biol. Chem., 272(17):11510-7, 1997.

Baughman, K., Cardiology Clinics, 13: 27-34, 1995.

Benvenisty and Neshif, “Direction introduction of genes into rats and expression of the genes”, Proc. Nat'l Acad. Sci. USA, 83:9551-9555, 1986.

Berkhout et al., “Tat trans-activates the human immunodeficiency virus through a nascent RNA target,” Cell, 59:273, 1989.

Bhavsar, Brand, Yacoub, Barton, “Isolation and characterization of the human cardiac troponin I gene (TNNI3),” Genomics, 35(1): 11-23, 1996.

Biben and Harvey, “Homeodomain factor Nkx2-5 controls left/right asymmetric expression of bHLH bene eHand during murine heart development,” Genes Dev., 11:1357-1369, 1997.

Black and Olson, “Transcriptional control of muscle development by myocyte enhancer factor-2 (MEF2) proteins,” Annual Rev. Cell Dev. Biol., 14:167-196, 1998.

Blanar, Baldwin, Flavell, Sharp, “A gamma-interferon-induced factor that binds the interferon response sequence of the MHC class I gene, H-2 Kb,” EMBO J., 8(4):1139-44, 1989.

Bodine and Ley, “An enhancer element lies 3′ to the human A gamma globin gene,” EMBO J, 6:2997, 1987.

Boshart, Weber, Jahn, Dorsch-Hasler, Fleckenstein, and Schaffner, “A very strong enhancer is located upstream of an immediate early gene of human cytomegalovirus,” Cell, 41:521, 1985.

Bosher and Labouesse, “RNA interference; genetic wand and genetic watchdog,” Nat. Cell Biol., 2:E31-E36, 2000.

Bosze, Thiesen, and Chamay, “A transcriptional enhancer with specificity for erythroid cells is located in the long terminal repeat of the friend murine leukemia virus,” EMBO J., 5:1615, 1986.

Bour, O'Brien, Lockwood, Goldstein, Bodmer, Taghert, Abmayr, Nguyen, “Drosophila MEF2, a transcription factor that is essential for myogenesis,” Gene Devel., 9:730-741, 1995.

Braddock, Chambers, Wilson, Esnouf, Adams, Kingsman, and Kingsman, “HIV-I Tat activates presynthesized RNA in the nucleus,” Cell, 58:269, 1989.

Braunwald, E. (ed), In: “Heart Disease,” W. B. Saunders, Philadelphia, page 426, 1988.

Brinster, Chen, Trumbauer, Yagle, Palmiter, “Factors affecting the efficiency of introducing foreign DNA into mice by microinjecting eggs,” Proc Nat'l Acad Sci USA, 82(13):4438-4442, 1985.

Bulla and Siddiqui, “The hepatitis B virus enhancer modulates transcription of the hepatitis B virus surface-antigen gene from an internal location,” J. Virol., 62:1437, 1986.

Campbell and Villarreal, “Functional analysis of the individual enhancer core sequences of polyoma virus: cell-specific uncoupling of DNA replication from transcription,” Mol. Cell. Biol., 8:1993, 1988.

Campbell, In: Monoclonal Antibody Technology, Laboratory Techniques in Biochemistry and Molecular Biology, Vol. 13, Burden and Von Knippenberg, Eds. pp. 75-83, Amsterdam, Elseview, 1984.

Campere and Tilghman, “Postnatal repression of the α-fetoprotein gene is enhancer independent,” Genes and Dev., 3:537, 1989.

Campo, Spandidos, Lang, Wilkie, “Transcriptional control signals in the genome of bovine papilloma virus type 1,” Nature, 303:77, 1983.

Capaldi, Bell, Branchek, “Changes in order of migration of polypeptides in complex III and cytochrome C oxidase under different conditions of SDS polyacrylamide gel electrophoresis,” Biochem. Biophys. Res. Comm., 76:425-433, 1977.

Celander and Haseltine, “Glucocorticoid regulation of murine leukemia virus transcription elements is specified by determinants within the viral enhancer region,” J Virology, 61:269, 1987.

Celander, Hsu, and Haseltine, “Regulatory Elements Within the Murine Leukemia Virus Enhancer Regions Mediate Glucocorticoid Responsiveness,” J. Virology, 62:1314, 1988.

Chandler, Maler, and Yamamoto, “DNA Sequences Bound Specifically by Glucocorticoid Receptor in vitro Render a Heterlogous Promoter Hormone Responsive in vivo,” Cell, 33:489, 1983.

Chang and Karin, “Mammalian MAP kinase signalling cascades,” Nature, 410(6824):37-40, 2001.

Chang et al, “Foreign gene delivery and expression in hepatocytes using a hepatitis B virus vector”, Hepatology, 14:124A, 1991.

Chang, Erwin, and Lee, “Glucose-regulated Protein (GRP94 and GRP78) Genes Share Common Regulatory Domains and are Coordinately Regulated by Common Trans-acting Factors,” Mol. Cell. Biol., 9:2153, 1989.

Chatterjee, Lee, Rentoumis, and Jameson, “Negative Regulation of the Thyroid-Stimulating Hormone Alpha Gene by Thyroid Hormone: Receptor Interaction Adjacent to the TATA Box,” Proc Nat'l Acad. Sci. U.S.A., 86:9114, 1989.

Chen and Okayama, “High-efficiency transfection of mammalian cells by plasmid DNA”, Mol. Cell Biol., 7:2745-2752, 1987.

Chen, Kerr, Chang, Honjo, Khalili, “Evidence for regulation of transcription and replication of the human neurotropic virus JCV genome by the human S9mu)bp-2 protein in glial cells,” Gene, 185:55-62, 1997.

Choi, Chen, Kriegler, and Roninson, “An altered pattern of cross-resistance in multi-drug-resistant human cells results from spontaneous mutations in the mdr-1 (p-glycoprotein) gene,” Cell, 53:519, 1988.

Clerk, A. & Sugden, P. H. (1999) Am. J. Cardiol. 83, 64H-69H.

Coffin, Retroviridae and Their Replication. In: Virology, Fields et al., eds., Raven Press, New York, pp. 1437-1500, 1990.

Cohen et al., “A repetitive sequence element 3′ of the human c-Ha-ras1 gene has enhancer activity”, J. Cell. Physiol., 5:75, 1987

Cook et al., “In vitro splicing of the ribosomal RNA precursor of Tetrahymena: involvement of a guanosine nucleotide in the excision of the intervening sequence,” Cell, 27:487-496, 1981.

Costa, Lai, Grayson, and Darnell, “The Cell-Specific Enhancer of the Mouse Transthyretin (Prealbumin) Gene Binds a Common Factor at One Site and a Liver-Specific Factor(s) at Two Other Sites,” Mol. Cell. Biol., 8:81, 1988.

Couch et al., “Immunization with types 4 and 7 adenovirus by selective infection of the intestinal tract,” Am. Rev. Resp. Dis., 88:394-403, 1963.

Coupar et al., “A general method for the construction of recombinant vaccinia virus expressing multiple foreign genes”, Gene, 68:1-10, 1988.

Cripe, Haugen, Turk, Tabatabai, Schmid, Durst, Gissmann, Roman, and Turek, “Transcriptional Regulation of the Human Papilloma Virus-16 E6-E7 Promoter by a Keratinocyte-Dependent Enhancer, and by Viral E2 Trans-Activator and Repressor Gene Products: Implications for Cervical Carcinogenesis,” EMBO J, 6:3745, 1987.

Cui, Hagan, Zhang, Peltz, “Identification and characterization of genes that are required for the accelerated degradation of mRNAs containing a premature translational termination codon,” Genes Devel., 9:423-436, 1995.

Culotta and Hamer, “Fine Mapping of a Mouse Metallothionein Gene Metal-Response Element,” Mol. Cell. Biol., 9:1376, 1989.

Czaplinski, Weng, Hagan, Peltz, “Purification and characterization of the Upfl protein: a factor involved in translation and mRNA degradation,” Rna, 1:610-623, 1995.

Dandolo, Blangy, and Kamen, “Regulation of Polyma Virus Transcription in Murine Embryonal Carcinoma Cells,” J. Virology, 47:55, 1983.

De la Cruz, Kressler, Linder, “Undwinding RNA in Saccharomyces cerevisiae, DEAD-box proteins and related families,” Trends in Biochem. Sciences, 24:192-198, 1999.

Dehaan, In Organogenesis, Dehaan and Ursprung (Eds.), Holt, Rinehart & Winston, New York, 377-419, 1965.

DeLuca, McCarthy, Schaffer. “Isolation and characterization of deletion mutants of herpes simplex virus type 1 in the gene encoding immediate-early regulatory protein ICP4,” J. Virol., 56(2):558-570, 1985.

DeMarini, Winey, Ursic, Webb, Culbertson, “SEN1, a positive effector of tRNA-splicing endonuclease in Saccharomyces cerevisiae,” Molecular Cellular Biol., 12:2154-2164, 1992.

Derby, Sena-Esteves, Breakefield, Corey, “Gene transfer into the mammalian inner ear using HSV-1 and vaccinia virus vectors,” Hear Res, 134(1-2):1-8, 1999.

Deschamps, Meijlink, and Verma, “Identification of a Transcriptional Enhancer Element Upstream From the Proto-Oncogene Fos,” Science, 230:1174, 1985.

Dubensky et al., “Direct transfection of viral and plasmid DNA into the liver or spleen of mice”, Proc. Nat'l Acad. Sci. USA, 81:7529-7533, 1984.

Edbrooke, Burt, Cheshire, and Woo, “Identification of cis-acting sequences responsible for phorbol ester induction of human serum amyloid a gene expression via a nuclear-factor-kappa β-like transcription factor,” Mol. Cell. Biol., 9:1908, 1989.

Edlund, Walker, Barr, and Rutter, “Cell-specific expression of the rat insulin gene: evidence for role of two distinct 5′ flanking elements,” Science, 230:912, 1985.

Edmondson, Lyons, Martin, Olson, “Mef2 gene expression marks the cardiac and skeletal muscle lineages during mouse embryogenesis,” Development, 120:1251-1263, 1994.

European Patent App. No. 0273085

Fechheimer, Boylan, Parker, Sisken, Patel and Zimmer, “Transfection of mammalian cells with plasmid DNA by scrape loading and sonication loading,” Proc Nat'l. Acad. Sci. USA 84:8463-8467, 1987

Feng and Holland, “HIV-I Tat Trans-Activation Requires the Loop Sequence Within Tar,” Nature, 334:6178, 1988.

Ferkol et al., “Regulation of the phosphoenolpyruvate carboxykinase/human factor 1×gene introduced into the livers of adult rats by receptor-mediated gene transfer”, FASEB J., 7:1081-1091, 1993.

Firak and Subramanian, “Minimal Transcription Enhancer of Simian Virus 40 is a 74-Base-Pair Sequence that Has Interacting Domains,” Mol. Cell. Biol., 6:3667, 1986.

Fire et al., Nature, 391:806-811, 1998.

Firulli, McFadden, Lin, Srivastava, Olson, “Heart and extra-embryonic mesodermal defects in mouse embryos lacking the bHLH transcription factor Handl,” Nature Gene., 18:266-270.

Fishman and Olson, “Parsing the heart: genetic modules for organ assembly,” Cell, 91:153-156, 1997.

Foecking and Hofstetter, “Powerful and Versatile Enhancer-Promoter Unit for Mammalian Expression Vectors,” Gene, 45(1):101-105, 1986.

Forster and Symons, “Self-cleavage of plus and minus RNAs of a virusoid and a structural model for the active sites,” Cell, 49:211-220, 1987.

Fraley, Fomari, Kaplan, “Entrapment of a bacterial plasmid in phospholipid vesicles:potential for gene transfer,” Proc Nat'l. Acad. Sci. USA 76:3348-3352, 1979.

Franz, Brem, Katus, Klingel, Hofschneider, Kandolf, “Characterization of a cardiac-selective and developmentally upregulated promoter in transgenic mice,” Cardoscience, 5(4):235-43, 1994.

Freifelder, Physical Biochemistry Applications to Biochemistry and Molecular Biology, 2nd ed. Wm. Freeman and Co., New York, N.Y., 1982.

Frey, N., Richardson, J. A., & Olson, E. N., Proc. Nat'l Acad. Sci. USA 97, 14632-14637, 2000.

Friedmann, “Progress toward human gene therapy”, Science, 244:1275-1281, 1989.

Fujita, Shibuya, Hotta, Yamanishi, and Taniguchi, “Interferon-Beta Gene Regulation: Tandemly Repeated Sequences of a Synthetic 6-bp Oligomer Function as a Virus-Inducible Enhancer,” Cell, 49:357, 1987.

Garrido, Carnicero, Lim, Schimmang, “Differential effects on the survival of neuronal and non-neuronal cells after infection by herpes simplex virus type 1 mutants,” J Neurovirol, 5(3):280-8, 1999.

Gefter, Margulies, Scharff, “A simple method for polyethylene glycol-promoted hybridization of mouse myeloma cells,” Somatic Cell Genet., 3:231-236, 1977.

Gerlach et al., “Construction of a plant disease resistance gene from the satellite RNA of tobacco rinspot virus,” Nature (London), 328:802-805, 1987.

Ghosh and Bachhawat, Targeting of Liposomes to Hepatocytes. In: Liver Diseases, Targeted Diagnosis and Therapy Using Specific Receptors and Ligands. Wu et al, eds., Marcel Dekker, New York, pp. 87-104, 1991.

Ghosh-Choudhury et al., “Protein IX, aminor component of the human adenovirus capsid, is essential for the packaging of full-length genomes,” EMBO J, 6:1733-1739, 1987.

Gilles, Morris, Oi, and Tonegawa, “A tissue-specific transcription enhancer element is located in the major intron of a rearranged immunoglobulin heavy-chain gene,” Cell, 33:717, 1983.

Gloss, Bernard, Seedorf, and Klock, “The Upstream Regulatory Region of the Human Papilloma Virus-16 Contains an E2 Protein-Independent Enhancer Which is Specific for Cervical Carcinoma Cells and Regulated by Glucocorticoid Hormones,” EMBO J., 6:3735, 1987.

Godbout, Ingram, and Tilghman, “Fine-Structure Mapping of the Three Mouse Alpha-Fetoprotein Gene Enhancers,” Mol. Cell. Biol., 8:1169, 1988.

Goding, 1986, In: Monoclonal Antibodies: Principles and Practice, 2d ed., Academic Press, Orlando, Fla., pp. 60-61, and 71-74, 1986.

Gomez-Foix et al., “Adenovirus-mediated transfer of the muscle glycogen phosphorylase gene into hepatocytes confers altered regulation of glycogen,” J. Biol. Chem., 267:25129-25134, 1992.

Goodboum and Maniatis, “Overlapping Positive and Negative Regulatory Domains of the Human β-Interferon Gene,” Proc. Nat'l Acad. Sci. USA, 85:1447, 1988.

Goodboum, Burstein, and Maniatis, “The Human Beta-Interferon Gene Enhancer is Under Negative Control,” Cell, 45:601, 1986.

Gopal, “Gene transfer method for transient gene expression, stable transfection, and cotransfection of suspension cell cultures”, Mol. Cell Biol., 5:1188-1190, 1985.

Gopal-Srivastava, Haynes, Piatigorsky, “Regulation of the murine αB-crystallin/small heat shock protein gene in cardiac muscle,” Muscle Cell. Biol., 15:7081-7090, 1995.

Graham and Prevec, In: Methods in Molecular Biology: Gene Transfer and Expression Protocol, E. J. Murray, ed., Humana Press, Clifton, N.J., 7:109-128, 1991.

Graham and van der Eb, “A new technique for the assay of infectivity of human adenovirus 5 DNA”, Virology, 52:456-467, 1973.

Graham et al., “Characteristics of a human cell line transformed by DNA from human adenovirus type 5”, J. Gen. Virol., 36:59-72, 1977.

Greene, Bohnlein, and Ballard, “HIV-1, and Normal T-Cell Growth: Transcriptional Strategies and Surprises,” Immunology Today, 10:272, 1989.

Grishok et al., Science, 287:2494-2497, 2000.

Grosschedl and Baltimore, “Cell-Type Specificity of Immunoglobulin Gene Expression is Regulated by at Least Three DNA Sequence Elements,” Cell, 41:885, 1985.

Grunhaus and Horwitz, “Adenovirus as cloning vector”, Seminar in Virology, 3:237-252, 1992.

Gulley, Zhang, Gascoyne, DuPont, Banks, Cho, Huang, Montalvo, “Translocations of 11q13 in mantle cell lymphoma fail to disrupt the S mu bp-2 gene,” Hematopathology Molecular Hematology, 11:1-11, 1997.

Han and Prywes, “Regulatory role of MEF2D in serum induction of the c-jun promoter,” Molecular Cellular Biology, 15:2907-2915, 1995.

Harland and Weintraub, “Translation of mammalian mRNA injected into Xenopus oocytes is specifically inhibited by antisense RNA”, J. Cell Biol., 101:1094-1099, 1985.

Harlow and Lane, Antibodies: A Laboratory manual, Cold Spring Harbor Laboratory, 1988.

Harvey, Dev. Biol. 178:203-216, 1996.

Haslinger and Karin, “Upstream Promoter Element of the Human Metallothionein-II Gene Can Act Like an Enhancer Element,” Proc Nat'l Acad. Sci. U.S.A., 82:8572, 1985.

Hauber and Cullen, “Mutational Analysis of the Trans-Activiation-Responsive Region of the Human Immunodeficiency Virus Type I Long Terminal Repeat,” J. Virology, 62:673, 1988.

Hayashi, Itabashi, Moriyama, “D-aspartate is present in human retinoblastoma Y79 cells,” Neurosci Lett, 267(1):37-40, 1999.

Hen, Borrelli, Fromental, Sassone-Corsi, and Chambon, “A Mutated Polyoma Virus Enhancer Which is Active in Undifferentiated Embryonal Carcinoma Cells is not Repressed by Adenovirus-2 E1A Products,” Nature, 321:249, 1986.

Hensel, Meichle, Pfizenmaier, and Kronke, “PMA-Responsive 5′Flanking Sequences of the Human TNF Gene,” Lymphokine Res., 8:347, 1989.

Hermonat and Muzycska, “Use of adenoassociated virus as a mammalian DNA cloning vector:

Transduction of neomycin resistance into mammalian tissue culture cells”, Proc. Nat'l Acad. Sci. USA, 81:6466-6470, 1984.

Hersdorffer et al., “Efficient gene transfer in live mice using a unique retroviral packaging line,” DNA Cell Biol., 9:713-723, 1990.

Herz and Gerard, “Adenovirus-mediated transfer of low density lipoprotein receptor gene acutely accelerates cholesterol clearance in normal mice,” Proc. Nat'l. Acad. Sci. USA 90:2812-2816, 1993.

Hirochika, Browker, and Chow, “Enhancers and Trans-Acting E2 Transcriptional Factors of Papilloma Viruses,” J. Virol., 61:2599, 1987.

Hirsch, Gaugler, Deagostini-Bauzin, Bally-Cuif, and Gordis, “Identification of Positive and Negative Regulatory Elements Governing Cell-Type-Specific Expression of the Neural-Cell-Adhesion-Molecule Gene,” Mol. Cell. Biol., 10:1959, 1990.

Holbrook, Gulino, and Ruscetti, “cis-Acting Transcriptional Regulatory Sequences in the Gibbon Ape Leukemia Virus (GALV) Long Terminal Repeat,” Virology, 157:211, 1987.

Horlick and Benfield, “The Upstream Muscle-Specific Enhancer of the Rat Muscle Creatine Kinase Gene is Composed of Multiple Elements,” Mol. Cell. Biol., 9:2396, 1989.

Horwich, et al., “Synthesis of hepadnavirus particles that contain replication-defective duck hepatitis B virus genomes in cultured HuH7 cells”, J. Virol., 64:642-650, 1990.

Huang et al, “A cellular protein that competes with SV40 antigen for binding to the retinoblastoma gene product,” Nature, 350:160-162, 1991.

Huard, Krisky, Oligino, Marconi, Day, Watkins, Glorioso, “Gene transfer to muscle using herpes simplex virus-based vectors,” Neuromuscul Disord, 7(5):299-313, 1997.

Hug, Costas, Staeheli, Aebi, and Weissmann, “Organization of the Murine Mx Gene and Characterization of its Interferon- and Virus-Inducible Promoter,” Mol. Cell. Biol., 8:3065, 1988.

Hwang, Lim, and Chae, “Characterization of the S-Phase-Specific Transcription Regulatory Elements in a DNA-Replication-Independent Testis-Specific H2B (TH2B) Histone Gene,” Mol. Cell. Biol., 10:585, 1990.

Imagawa, Chiu, and Karin, “Transcription Factor AP-2 Mediates Induction by Two Different Signal-Transduction Pathways: Protein Kinase C and cAMP,” Cell, 51:251, 1987.

Imbra and Karin, “Phorbol Ester Induces the Transcriptional Stimulatory Activity of the SV40 Enhancer,” Nature, 323:555, 1986.

Imler, Lemaire, Wasvlyk, and Waslyk, “Negative Regulation Contributes to Tissue Specificity of the Immunoglobulin Heavy-Chain Enhancer,” Mol. Cell. Biol, 7:2558, 1987.

Imperiale and Nevins, “Adenovirus 5 E2 Transcription Unit: an E1A-Inducible Promoter with an Essential Element that Functions Independently of Position or Orientation,” Mol. Cell. Biol., 4:875, 1984.

Innis et al., “DNA sequencing with Thermus aquaticus DNA polymerase and direct sequencing of polymerase chain reaction-amplified DNA,” Proc Natl Acad Sci USA. 85(24):9436-9440, 1988.

Jakobovits, Smith, Jakobovits, and Capon, “A Discrete Element 3′ of Human Immunodeficiency Virus 1 (HIV-1) and HIV-2 mRNA Initiation Sites Mediates Transcriptional Activation by an HIV Trans-Activator,” Mol Cell. Biol., 8:2555, 1988.

Jameel and Siddiqui, “The Human Hepatitis B Virus Enhancer Requires Transacting Cellular Factor(s) for Activity,” Mol. Cell. Biol., 6:710, 1986.

Jaynes, Johnson, Buskin, Gartside, and Hauschka, “The Muscle Creatine Kinase Gene is Regulated by Multiple Upstream Elements, Including a Muscle-Specific Enhancer,” Mol. Cell. Biol., 8:62, 1988.

Johnson et al., Peptide Turn Mimetics” IN: Biotechnology And Pharmacy, Pezzuto et al., eds., Chapman and Hall, New York, 1993.

Johnson, Wold, and Hauschka, “Muscle creatine kinase sequence elements regulating skeletal and cardiac muscle expression in transgenic mice,” Mol. Cell. Biol., 9:3393, 1989.

Jones and Shenk, “Isolation of deletion and substitution mutants of adenovirus type 5,” Cell, 13:181-188, 1978.

Joyce, “RNA evolution and the origins of life,” Nature, 338:217-244, 1989.

Kadesch and Berg, “Effects of the Position of the Simian Virus 40 Enhancer on Expression of Multiple Transcription Units in a Single Plasmid,” Mol. Cell. Biol., 6:2593, 1986.

Kaneda et al., “Increased expression of DNA cointroduced with nuclear protein in adult rat liver”, Science, 243:375-378, 1989.

Karin, Haslinger, Heguy, Dietlin, and Cooke, “Metal-Responsive Elements Act as Positive Modulators of Human Metallothionein-IIA Enhancer Activity,” Mol. Cell. Biol., 7:606, 1987.

Karlsson et al., EMBO J., 5:2377-2385, 1986.

Katinka, Vasseur, Montreau, Yaniv, and Blangy, “Polyoma DNA Sequences Involved in the Control of Viral Gene Expression in Murine Embryonal Carcinoma Cells,” Nature, 290:720, 1981.

Katinka, Yaniv, Vasseur, and Blangy, “Expression of Polyoma Early Functions in Mouse Embryonal Carcinoma Cells Depends on Sequence Rearrangements in the Beginning of the Late Region,” Cell, 20:393, 1980.

Kato et al., “Expression of hepatitis β virus surface antigen in adult rat liver. Co-introduction of DNA and nuclear protein by a simplified liposome method,” J. Biol. Chem., 266(6):3361-3364, 1991.

Kawamoto, Makino, Niw, Sugiyama, Kimura, Anemura, Nakata, and Kakunaga, “Identification of the Human Beta-Actin Enhancer and its Binding Factor,” Mol. Cell. Biol., 8:267, 1988.

Kelly, Alonso, Tajbakhsh, Cossu, Buckingham, “Myosin light chain 3F regulatory sequences confer regionalized cardiac and skeletal muscle expression in transgenic mice,” J. Cell Biol., 129(2):383-96, 1995.

Ketting et al., Cell, 99:133-141, 1999.

Kiledjian, Su, Kadesch, “Identification and characterization of two functional domains within the murine heavy-chain enhancer,” Mol. Cell. Biol., 8:145, 1988.

Kim and Cook, “Three dimensional model of the active site of the self-splicing rRNA precursor or Tetrahymena,” Proc. Nat'l Acad. Sci. USA, 84:8788-8792, 1987.

Kim, Choe, Seo, “The sen1(+) gene of Schizosaccharomyces pombe, a homologue of budding yeast SEN1, encodes an RNA and DNA helicase,” Biochemistry, 38:14697-14710, 1999.

Kimura, Abe, Suzuki, Ogawa, Yoshioka, Kaname, Miike, Yamamura, “A 900 bp genomic region from the mouse dystrophin promoter directs lacZ reporter expression only to the right heart of transgenic mice,” Dev. Growth Differ., 39(3):257-65, 1997.

Klamut, Gangopadyhay, Worton, and Ray, “Molecular and Functional Analysis of the Muscle-Specific Promoter Region of the Duchenne Muscular Dystrophy Gene,” Mol. Cell. Biol., 10:193, 1990.

Klein et al., “High-velocity microprojectiles for delivering nucleic acids into living cells”, Nature, 327:70-73, 1987.

Koch, Benoist, and Mathis, “Anatomy of a new β-cell-specific enhancer,” Mol. Cell. Biol., 9:303, 1989.

Kodama, et al., Cir. Res. 81:656-663, 1997.

Kohler and Milstein, “Continuous cultures of fused cells secreting antibody of predefined specificity,” Nature, 256:495-497, 1975.

Kohler and Milstein, “Derivation of specific antibody-producing tissue culture and tumor lines by cell fusion,” Eur. J. Immunol., 6:511-519, 1976.

Kohut, Davis, Jackson, Jani, Ghaffar, Mayer, Essig, “Exercise effects on IFN-beta expression and viral replication in lung macrophages after HSV-1 infection,” Am J Physiol, 275(6 Pt 1):L1089-94, 1998.

Kooby, Carew, Halterman, Mack, Bertino, Blumgart, Federoff, Fong, “Oncolytic viral therapy for human colorectal cancer and liver metastases using a multi-mutated herpes simplex virus type-1 (G207),” FASEB J, 13(11):1325-34, 1999.

Kriegler and Botchan, “Enhanced transformation by a simian virus 40 recombinant virus containing a Harvey murine sarcoma virus long terminal repeat,” Mol. Cell. Biol. 3:325, 1983.

Kriegler and Botchan, In: Eukaryotic Viral Vectors, Y. Gluzman, ed., Cold Spring Harbor: Cold Spring Harbor Laboratory, NY, 1982.

Kriegler et al., “Promoter substitution and enhancer augmentation increases the penetrance of the sv40a gene to levels comparable to that of the harvey murine sarcoma virus ras gene in morphologic transformation,” In: Gene Expression, Alan Liss (Ed.), Hamer and Rosenberg, New York, 1983.

Kriegler et al., “Transformation Mediated by the SV40 T Antigens: Separation of the Overlapping SV40 Early Genes with a Retroviral Vector,” Cell, 38:483, 1984.

Kriegler et al., “Viral Integration and Early Gene Expression Both Affect the Efficiency of SV40 Transformation of Murine Cells: Biochemical and Biological Characterization of an SV40 Retrovirus,” In: Cancer Cells 2/Oncogenes and Viral Genes, Van de Woude et al. eds, Cold Spring Harbor: Cold Spring Harbor Laboratory, 1984.

Kriegler, Perez, Defay, Albert and Liu, “A Novel Form of TNF/Cachectin Is a Cell-Surface Cytotoxix Transmembrane Protein: Ramifications for the Complex Physiology of TNF,” Cell, 53:45, 1988.

Krisky, Marconi, Oligino, Rouse, Fink, Cohen, Watkins, Glorioso, “Development of herpes simplex virus replication-defective multigene vectors for combination gene therapy applications,” Gene Ther, 5(11):1517-1530, 1998.

Krisky, Wolfe, Goins, Marconi, Ramakrishnan, Mata, Rouse, Fink, Glorioso, “Deletion of multiple immediate-early genes from herpes simplex virus reduces cytotoxicity and permits long-term gene expression in neurons,” Gene Ther, 5(12):1593-1603, 1998.

Kuhl, De La Fuenta, Chaturvedi, Parinool, Ryals, Meyer, and Weissman, “Reversible Silencing of Enhancers by Sequences Derived From the Human IFN-alpha Promoter,” Cell, 50:1057, 1987.

Kuisk, Li, Tran, Capetanaki, “A single MEF2 site governs desmin transcription in both heart and skeletal muscle during mouse embryogenesis,” Developmental Biology, 174:1-13, 1996.

Kunz, Zimmerman, Heisig, and Heinrich, “Identification of the Promoter Sequences Involved in the Interleukin-6-Dependent Expression of the Rat Alpha-2-Macroglobulin Gene,”Nucl. Acids Res., 17:1121, 1989.

Kuo, Morrisey, Anandappa, Sigrist, Lu, Parmacek, Soudais, Leiden, “GATA4 transcription factor is required for ventral morphogenesis and heart tube formation,” Genes Development, 11:1048-1060, 1997.

Kyte and Doolittle, “A simple method for displaying the hydropathic character of a protein,” J. Mol. Biol., 157(1):105-132, 1982.

Lachmann and Efstathiou, “Use of herpes simplex virus type 1 for transgene expression within the nervous system,” Clin Sci (Lond), 96(6):533-41, 1999.

LaPointe, Wu, Greenberg, Gardner, “Upstream sequences confer atrial-specific expression on the human atrial natriuretic factor gene.” J. Biol. Chem., 263(19):9075-8, 1988.

Larsen, Harney, and Moore, “Repression medaites cell-type-specific expression of the rat growth hormone gene,” Proc Nat'l Acad. Sci. USA., 83:8283, 1986.

Laspia, Rice, and Mathews, “HIV-1 Tat protein increases transcriptional initiation and stabilizes elongation,” Cell, 59:283, 1989.

Latimer, Berger, and Baumann, “Highly conserved upstream regions of the α₁-antitrypsin gene in two mouse species govern liver-specific expression by different mechanisms,” Mol. Cell. Biol., 10:760, 1990.

Le Gal La Salle et al., “An adenovirus vector for gene transfer into neurons and glia in the brain,” Science, 259:988-990, 1993.

Lee, Mulligan, Berg, and Ringold, “Glucocorticoids Regulate Expression of Dihydrofolate Reductase cDNA in Mouse Mammary Tumor Virus Chimaeric Plasmids,” Nature, 294:228, 1981.

Leeds, Peltz, Jacobson, Culbertson, “The product of the yeast UPF1 gene is required for rapid turnover of mRNAs containinga premature translational termination codon,” Genes Development, 5:2303-2314, 1991.

Lelivelt and Culbertson, “Yeast Upf proteins required for RNA surveillance affect global expression of the eyast transcriptome,” Molecular Cellular Biology, 19:6710-6719, 1999.

Leor et al., Circulation 94(Suppl. II):332-336, 1996.

Levinson, Khoury, VanDeWoude, and Gruss, “Activation of SV40 Genome by 72-Base-Pair Tandem Repeats of Moloney Sarcoma Virus,” Nature, 295:79, 1982.

Levrero et al., “Defective and nondefective adenovirus vectors for expressing foreign genes in vitro and in vivo,” Gene, 101:195-202, 1991.

Li, J. M. & Grooks, G., Eur. Heart J 20, 406-420, 1999.

Lilly, Galewsky, Firulli, Schulz, Olson, “D-MEF2: a MADs box transcription factor expressed in differentiating mesoderm and muscle cell lineages during Drosophila embryogenesis,” Proc. Nat'l Acad. Sci. USA, 91:5662-5666, 1994.

Lilly, Zhao, Ranganayakulu, Paterson, Schulz, Olson “Requirement of MADS domain transcription factor D-MEF2 for muscle formation in Drosophila,” Science, 267:688-693, 1995.

Lin & Avery, Nature, 402:128-129, 1999.

Lin, Cross, Halden, Dragos, Toledano, and Leonard, “Delineation of an enhancerlike positive regulatory element in the interleukin-2 receptor α-chain gene,” Mol. Cell. Biol., 10:850, 1990.

Lin, Schwarz, Bucana, Olson, “Control of mouse cardiac morphogenesis and myogenesis by transcription factor MEF2C,” Science, 276:1404-1407, 1997.

Liu, Z. P., Nakagawa, O., Nakagawa, M., Yanagisawa, H., Passier, R., Richardson, J. A., Srivastava, D., & Olson, E. N. Dev. Biol. 234, 497-509, 2001.

Luria, Gross, Horowitz, and Givol, “Promoter Enhancer Elements in the Rearranged Alpha-Chain Gene of the Human T-Cell Receptor,” EMBO J., 6:3307, 1987.

Lusky and Botchan, “Transient Replication of Bovine Papilloma Virus Type 1 Plasmids: cis and trans Requirements,” Proc Nat'l Acad. Sci. U.S.A., 83:3609, 1986.

Lusky, Berg, Weiher, and Botchan, “Bovine Papilloma Virus Contains an Activator of Gene Expression at the Distal End of the Early Transcription Unit,” Mol. Cell. Biol. 3:1108, 1983.

Macejak and Samow, “Internal initiation of translation mediated by the 5′ leader of a cellular mRNA,” Nature, 353:90-94, 1991.

Majors and Varmus, “A Small Region of the Mouse Mammary Tumor Virus Long Terminal Repeat Confers Glucocorticoid Hormone Regulation on a Linked Heterologous Gene,” Proc. Nat'l Acad. Sci. USA, 80:5866, 1983.

Makino et. al., J. Clin. Invest., 103:697-705, 1999.

Manipulating the Mouse Embryo: A Laboratory Manual, 2nd ed., Hogan et al., eds., Cold Spring Harbor Laboratory Press, 1994.

Mann et al., “Construction of a retrovirus packaging mutant and its use to produce helper-free defective retrovirus”, Cell, 33:153-159, 1983.

Markowitz et al., “A safe packaging line for gene transfer: Separating viral genes on two different plasmids,” J. Virol., 62:1120-1124, 1988.

McNeall, Sanchez, Gray, Chesterman, and Sleigh, “Hyperinducible Gene Expression From a Metallotionein Promoter Containing Additional Metal-Responsive Elements,” Gene, 76:81, 1989.

Merrifield, “Solid phase synthesis,” Science, 232: 341-347, 1986.

Michel and Westhof, “Modeling of the three-dimensional architecture of group I catalytic introns based on comparative sequence analysis,” J. Mol. Biol., 216:585-610, 1990.

Miksicek, Heber, Schmid, Danesch, Posseckert, Beato, and Schutz, “Glucocorticoid Responsiveness of the Transcriptional Enhancer of Moloney Murine Sarcoma Virus,” Cell, 46:203, 1986.

Molkentin and Olson, “GATA4: a novel transcriptional regulator of cardiac hypertrophy?” Circulation, 96:3833-3835, 1997.

Molkentin, J. D., Lu, J. R., Antos, C. L., Markham, B., Richardson, J., Robbins J., Grant, S. R., & Olson, E. N. (1998) Cell 93, 215-228.

Montgomery et al., Proc. Nat'l Acad. Sci. USA, 95:155-2-15507, 1998.

Mordacq and Linzer, “Co-localization of Elements Required for Phorbol Ester Stimulation and Glucocorticoid Repression of Proliferin Gene Expression,” Genes and Dev., 3:760, 1989.

Moreau, Hen, Wasylyk, Everett, Gaub, and Chambon, “The SV40 base-repair repeat has a striking effect on gene expression both in sv40 and other chimeric recombinants,” Nucl. Acids Res., 9:6047, 1981.

Moriuchi, Oligino, Krisky, Marconi, Fink, Cohen, Glorioso, “Enhanced tumor cell killing in the presence of ganciclovir by herpes simplex virus type 1 vector-directed coexpression of human tumor necrosis factor-alpha and herpes simplex virus thymidine kinase,” Cancer Res, 58(24):5731-5737, 1998.

Moss, Marshall, Moczydlowski, “Hypothesis for a serine proteinase-like domain at the COOH terminus of Slowpoke calcium-activated potassium channels,” J. Gen. Physiol. 108(6):473-84, 1996.

Musesing, Smith, and Capon, “Regulation of mRNA Accumulation by a Human Immunodeficiency Virus Trans-Activator Protein,” Cell, 48:691, 1987.

Nakagawa, Nakagawa, Richardson, Olson, Srivastava, “HRT1, HRT2, and HRT3: a new subclass of bHLH transcription factors marking specific cardiac, somitic, and pharyngeal arch segment,” Develop. Biol., 216:72-84, 1999.

Nakajima, Uchida, Anderson, Lee, Hurwitz, Parvin, Montminy, “RNA helicase A mediates association of CBP with RNA polymerase II,” Cell, 90:1107-1112, 1997.

Naya and Olson, “MEF2: a transcriptional target for signaling pathways controlling skeletal muscle growth and differentiation,” Curr. Opinion Cell Biol., 11:683-688, 1999.

Ng, Gunning, Liu, Leavitt, and Kedes, “Regulation of the Human Beta-Actin Promoter by Upstream and Intron Domains,” Nuc. Acids Res., 17:601, 1989.

Nguyen, Bodmer, Abmayr, McDermott, Spoerel, “D-mef2: a Drosophila mesoderm-specific MADS box-containing gene with a biphasic expresssion profile during embryogenesis,” Proc. Nat'l Acad. Sci. USA, 91:7520-7524, 1994.

Nicol, R. L., Frey, N., Pearson, G., Cobb, M., Richardson, J., & Olson, E. N., EMBO J. 20, 2757-2767, 2001.

Nicolas and Rubinstein, In: Vectors: A survey of molecular cloning vectors and their uses, Rodriguez and Denhardt, eds., Stoneham: Butterworth, pp. 494-513, 1988.

Nicolau and Sene, “Liposome-mediated DNA transfer in eukaryotic cells”, Biochim. Biophys. Acta, 721:185-190, 1982.

Nicolau et al., “Liposomes as carriers for in vivo gene transfer and expression,” Methods Enzymol., 149:157-176, 1987.

Nozato, T., Ito, H., Watanabe, M., Ono, Y., Adachi, S., Tanaka, H., Hiroe, M., Sunamori, M., & Marum, F., J. Mol. Cell. Cardiol. 33, 1493-1504, 2000.

Olson and Srivastava, “Molecular pathways controlling heart development,” Science, 272:671-676, 1996.

Ondek, Sheppard, and Herr, “Discrete Elements Within the SV40 Enhancer Region Display Different Cell-Specific Enhancer Activities,” EMBO J., 6:1017, 1987.

Ornitz, Hammer, Davison, Brinster, and Palmiter, “Promoter and enhancer elements from the rat elastase i gene function independently of each other and of heterologous enhancers,” Mol. Cell. Biol. 7:3466, 1987.

Palmiter, Chen, and Brinster, “Differential regulation of metallothionein-thymidine kinase fusion genes in transgenic mice and their offspring,” Cell, 29:701, 1982.

Pan, et al., Circ. Res. 81:611-617, 1997.

Paskind et al., “Dependence of moloney murine leukemia virus production on cell growth”, Virology, 67:242-248, 1975.

Passier, Xheng, Frey, Naya, Nicol, McKinsey, Overbeek, Richardson, Grant, Olson, “CaM kinase signaling induces cardiac hypertrophy and activates the MEF2 transcription factor in vivo,” J. Clin. Invest., 105(10):1395-406, 2000.

Pech, Rao, Robbins, and Aaronson, “Functional identification of regulatory elements within the promoter region of platelet-derived growth factor 2,” Mol. Cell. Biol., 9:396, 1989.

Pelletier and Sonenberg, “Internal initiation of translation of eukaryotic mRNA directed by a sequence derived from poliovirus RNA,” Nature, 334:320-325, 1988.

Perales, Ferkol, Beegen, Ratnoff, Hanson, “Gene transfer in vivo: sustained expression and regulation of genes introduced into the liver by receptor-targeted uptake,” Proc. Nat'l Acad. Sci. USA, 91(9):4086-4090, 1994.

Perez-Stable and Constantini, “Roles of fetal γ-globin promoter elements and the adult β-globin 3′ enhancer in the stage-specific expression of globin genes,” Mol. Cell. Biol., 10:1116, 1990.

Picard and Schaffner, “A lymphocyte-specific enhancer in the mouse immunoglobulin kappa gene,” Nature, 307:83, 1984.

Pignon, Vinatier, Fanen, Jonveaux, Tournilhac, Imbert, Rochant, Goossens, “Exhaustive analysis of the P53 gene coding sequence by denaturing gradient gel electrophoresis: application to the detection of point mutations in acute leukemias,” Hum. Mutat., 3: 126-132, 1994.

Pinkert, Omitz, Brinster, and Palmiter, “An albumin enhancer located 10 kb upstream functions along with its promoter to direct efficient, liver-specific expression in transgenic mice,” Genes and Dev., 1:268, 1987.

Plasterk and Ketting, “The silence of the genes,” Curr. Opin. Genet. Dev., 10:562-567, 2000.

Ponta, Kennedy, Skroch, Hynes, and Groner, “Hormonal Response Region in the Mouse Mammary Tumor Virus Long Terminal Repeat Can Be Dissociated From the Proviral Promoter and Has Enhancer Properties,” Proc. Nat'l Acad. Sci. U.S.A., 82:1020, 1985.

Porton, Zaller, Lieberson, and Eckhardt, “Immunoglobulin heavy-chain enhancer is required to maintain transfected .gamma.2a gene expression in a pre-b-cell line,” Mol. Cell. Biol., 10:1076, 1990.

Poser Impey, Trinh, Xia, Storm, “SRF-dependent gene expression is required for PI3-kinase-regulated cell proliferation,” EMBO J, 19(18):4955-66, 2000.

Potter et al., “Enhancer-dependent expression of human k immunoglobulin genes introduced into mouse pre-B lymphocytes by electroporation,” Proc. Nat'l Acad. Sci. USA, 81:7161-7165, 1984.

Queen and Baltimore, “Immunoglobulin gene transcription is activated by downstream sequence elements,” Cell, 35:741, 1983.

Quinn, Farina, Gardner, Krutzsch, and Levens, “Multiple components are required for sequence recognition of the ap1 site in the gibbon ape leukemia virus enhancer,” Mol. Cell. Biol., 9:4713, 1989.

Rabinovitch, Suarez-Pinzon, Strynadka, Ju, Edelstein, Brownlee, Korbutt, Rajotte, “Transfection of human pancreatic islets with an anti-apoptotic gene (bcl-2) protects beta-cells from cytokine-induced destruction,” Diabetes, 48(6): 1223-9, 1999.

Racher et al., Biotechnology Techniques, 9:169-174, 1995.

Ragot et al., “Efficient adenovirus-mediated transfer of a human minidystrophin gene to skeletal muscle of mdx mice,” Nature, 361:647-650, 1993.

Ranganayakulu, Zhao, Dokidis, Molentin, Olson, Schulz, “A series of mutations in the D-MEF2 transcription factor reveal multiple functions in larval and adult myogenesis in Drosophila, Dev. Biology, 171:169-181, 1995.

Redondo, Hata, Brocklehurst, and Krangel, “A T-Cell-Specific Transcriptional Enhancer Within the Human T-Cell Receptor .delta Locus,” Science, 247:1225, 1990.

Reinhold-Hurek and Shub, “Self-splicing introns in tRNA genes of widely divergent bacteria,” Nature, 357:173-176, 1992.

Reisman and Rotter, “Induced expression from the moloney murine leukemia virus long terminal repeat during differentiation of human myeloid cells is mediated through its transcriptional enhancer,” Mol. Cell. Biol., 9:3571, 1989.

Reiter, Alexander, Rodaway, Yelon, Pateint, Holder, Stainer, “Gata5 is required for the development of theart and endoderm in zebrafish,” Genes Develop., 13:2983-2995, 1999.

Renan, “Cancer genes: Current status, future prospects and applications in radiotherapy/oncology,” Radiother. Oncol., 19:197-218, 1990.

Resendez Jr., Wooden, and Lee, “Identification of highly conserved regulatory domains and protein-binding sites in the promoters of the rat and human genes encoding the stress-inducible 78-kilodalton glucose-regulated protein,” Mol. Cell. Biol., 8:4579, 1988.

Rich et al., “Development and analysis of recombinant adenoviruses for gene therapy of cystic fibrosis,” Hum. Gene Ther., 4:461-476, 1993.

Ridgeway, Mammalian Expression Vectors, In: Vectors: A Survey of Molecular Cloning Vectors and Their Uses, Rodriguez et al., eds., Stoneham: Butterworth, pp. 467-492, 1988.

Ripe, Lorenzen, Brenner, and Breindl, “Regulatory elements in the 5′ flanking region and the first intron contribute to transcriptional control of the mouse alpha-1-type collagen gene,” Mol. Cell. Biol., 9:2224, 1989.

Rippe, Brenner and Leffert, “DNA-mediated gene transfer into adult rat hepatocytes in primary culture,” Mol. Cell Biol., 10:689-695, 1990.

Rittling, Coutinho, Amarm, and Kolbe, “AP-1/jun-binding Sites Mediate Serum Inducibility of the Human Vimentin Promoter,” Nuc. Acids Res., 17:1619, 1989.

Rosen, Sodroski, and Haseltine, “The location of cis-acting regulatory sequences in the human t-cell lymphotropic virus type III (HTLV-111/LAV) long terminal repeat,” Cell, 41:813, 1988.

Rosenfeld, Siegfried, Yoshimura, Yoneyama, Fukayama, Stier, Paakko, Gilardi, Stratford-Perricaudet, Perricaudet, Jallat, Pavirani, Lecocq, Crystal, “Adenovirus-mediated transfer of a recombinant α1-antitrypsin gene to the lung epithelium in vivo,” Science, 252:431-434, 1991.

Rosenfeld, Yoshimura, Trapnell, Yoneyama, Rosenthal, Dalemans, Fukayama, Bargon, Stier, Stratford-Perricaudet, Perricaudet, Guggino, Pavirani, Lecocq, Crystal, “In vivo transfer of the human cystic fibrosis transmembrane conductance regulator gene to the airway epithelium,” Cell, 68:143-155, 1992.

Ross, Navankasattusas, Harvey, Chien, “An HF-1a/HF-1b/MEF-2 combinatorial element confers cardiac ventricular specificity and established an anterior-posterior gradient of expression,” Development, 122:1799-1809, 1996.

Roux et al., “A versatile and potentially general approach to the targeting of specific cell types by retroviruses: Application to the infection of human cells by means of major histocompatibility complex class I and class II antigens by mouse ecotropic murine leukemia virus-derived viruses”, Proc. Nat'l Acad. Sci. USA, 86:9079-9083, 1989.

Sakai, Helms, Carlstedt-Duke, Gustafsson, Rottman, and Yamamoto, “Hormone-Mediated Repression: A Negative Glucocorticoid-Response Element From the Bovine Prolactin Gene,” Genes and Dev., 2:1144, 1988.

Sambrook, Fritsch, Maniatis, Molecular Cloning: A Laboratory Manual, 2nd Ed., Cold Spring Harbor Press, Cold Spring Harbor, N.Y., 1989.

Sarver, et al, “Ribozymes as potential anti-HIV-1 therapeutic agents,” Science, 247:1222-1225, 1990.

Satake, Furukawa, and Ito, “Biological activities of oligonucleotides spanning the f9 point mutation within the enhancer region of polyoma virus DNA,” J. Virology, 62:970, 1988.

Scanlon et al., “Ribozyme-mediated cleavages of c-fos mRNA reduce gene expression of DNA synthesis enzymes and metallothionein,” Proc. Nat'l Acad. Sci. USA, 88:10591-10595, 1991.

Schaffner, Schirm, Muller-Baden, Wever, and Schaffner, “Redundancy of Information in Enhancers as a Principle of Mammalian Transcription Control,” J. Mol. Biol., 201:81, 1988.

Searle, Stuart, and Palmiter, “Building a metal-responsive promoter with synthetic regulatory elements,” Mol. Cell. Biol., 5:1480, 1985.

Sebastiani, Durocher, Gros, Nemer, Malo, “Localization of the Catf1 transcription factor gene to mouse chromosome 19,” Mammalian Genome, 6:147-148, 1995.

Sedmera, D., Pexieder, T., Vuillemin, M., Thompson, R. P., & Anderson, R. H., Anat. Rec. 258, 319-337, 2000.

Sharp and Marciniak, “HIV Tar: an RNA Enhancer?,” Cell, 59:229, 1989.

Sharp and Zamore, Science, 287:2431-2433, 2000.

Sharp, Genes Dev., 13:139-141, 1999.

Shaul and Ben-Levy, “Multiple Nuclear Proteins in Liver Cells are Bound to Hepatitis B Virus Enhancer Element and its Upstream Sequences,” EMBO J, 6:1913, 1987.

Sherman, Basta, Moore, Brown, and Ting, “Class II Box Consensus Sequences in the HLA-DRα. Gene: Transcriptional Function and Interaction with Nuclear Proteins,” Mol. Cell. Biol., 9:50, 1989. Siomi and Dreyfuss, “RNA-binding proteins as regulators of gene expression,” Curr. Opinion Genetics Dev., 7:345-353, 1997.

Sleigh and Lockett, “SV40 Enhancer Activation During Retinoic-Acid-Induced Differentiation of F9 Embryonal Carcinoma Cells,” J EMBO, 4:3831, 1985.

Sobue et al., 1999

Spalholz, Yang, and Howley, “Transactivation of a Bovine Papilloma Virus Transcriptional Regulatory Element by the E2 Gene Product,” Cell, 42:183, 1985.

Spandau and Lee, “Trans-Activation of Viral Enhancers by the Hepatitis B Virus X Protein,” J. Virology, 62:427, 1988.

Spandidos and Wilkie, “Host-Specificities of Papilloma Virus, Moloney Murine Sarcoma Virus and Simian Virus 40 Enhancer Sequences,” EMBO J., 2:1193, 1983.

Srivastava et. al., Cell, 56:607-617, 1995.

Srivastava, “HAND proteins: molecular mediators of cardiac development and congenital heart disease,” Trends in Cardiovascular Medicine, 9:11-18, 1999.

Srivastava, Cserjesi, Olson, “A subclass of bHLH proteins required for cardiac morphogenesis, Sciences, 270:1995-1999, 1995.

Srivastava, Thomas, Lin, Kirby, Brown, Olson, “Regulation of cardiac mesodermal and neural crest development by the bHLH transcription factor, dHAND,” Nature Genetics, 16:5477-5490, 1996.

Stephens and Hentschel, “The Bovine Papilloma Virus Genome and its Uses as a Eukaryotic Vector,” Biochem. J, 248:1, 1987.

Stewart and Young, Solid Phase Peptide Synthesis, 2d. ed., Pierce Chemical Co., 1984.

Stratford-Perricaudet and Perricaudet, Gene transfer into animals: the promise of adenovirus. In: Human Gene Transfer, O. Cohen-Haguenauer et al., eds., John Libbey Eurotext, France, pp. 51-61, 1991.

Stratford-Perricaudet et al., “Evaluation of the transfer and expression in mice of an enzyme-encoding gene using a human adenovirus vector”, Hum. Gene. Ther., 1:241-256, 1990.

Stuart, Searle, and Palmiter, “Identification of Multiple Metal Regulatory Elements in Mouse Metallothionein-I Promoter by Assaying Synthetic Sequences,” Nature, 317:828, 1985.

Sullivan and Peterlin, “Transcriptional Enhancers in the HLA-DQ Subregion,” Mol. Cell. Biol., 7:3315, 1987.

Suzuki, Piche, Kasono, Xiang, Gomez-Navarro, Moriuchi, Krisky, Oligino, Glorioso, Curiel, Curiel, “Efficient gene delivery into epstein-barr virus (EBV)-transformed human B cells mediated by replication-defective herpes simplex virus-1 (HSV-1): A gene therapy model for EBV-related B cell malignancy,” Biochem Biophys Res Commun, 252(3):686-90, 1998.

Swartzendruber and Lehman, “Neoplastic Differentiation: Interaction of Simian Virus 40 and Polyoma Virus with Murine Teratocarcinoma Cells,” J. Cell. Physiology, 85:179, 1975. Takebe et al., Mol. Cell. Biol., 8:466, 1988.

Tabaraet al., Cell, 99:123-132, 1999.

Tam et al., J. Am. Chem. Soc., 105:6442, 1983.

Tang et al., “A novel Gonadotropin-regulated Testicular RNA Helicase,” J. Biol. Chem. 274:37932-37940, 1999.

Tavernier, Gheysen, Duerinck, Can Der Heyden, and Fiers, “Deletion Mapping of the Inducible Promoter of Human IFN-beta Gene,” Nature, 301:634, 1983.

Taylor and Kingston, “E1A Trans-Activation of Human HSP70 Gene Promoter Substitution Mutants is Independent of the Composition of Upstream and TATA Elements,” Mol. Cell. Biol., 10:176, 1990.

Taylor and Kingston, “Factor Substitution in a Human HSP70 Gene Promoter: TATA-Dependent and TATA-Independent Interactions,” Mol. Cell. Biol., 10:165, 1990a.

Taylor, Solomon, Weiner, Paucha, Bradley, and Kingston, “Stimulation of the Human Heat-Shock Protein 70 Promoter in vitro by Simian Virus 40 Large T Antigen,” J. Biol. Chem., 264:15160, 1989.

Temin, Retrovirus vectors for gene transfer: Efficient integration into and expression of exogenous DNA in vertebrate cell genome. In: Gene Transfer, Kucherlapati R, ed., New York, Plenum Press, pp. 149-188, 1986.

Thiesen, Bosze, Henry, and Charnay, “A DNA Element Responsible for the Different Tissue Specificities of Friend and Moloney Retroviral Enhancers,” J. Virology, 62:614, 1988.

Top et al., “Immunization with live types 7 and 4 adenovirus vaccines. II. Antibody response and protective effect against acute respiratory disease due to adenovirus type 7,” J. Infect. Dis., 124:155-160, 1971.

Treisman, “Transient Accumulation of c-fos RNA Following Serum Stimulation Requires a Conserved 5′ Element and c-fos 3′ Sequences,” Cell, 42:889, 1986.

Tronche, Rollier, Herbomel, Bach, Cereghini, Weiss, and Yaniv, “Anatomy of the Rat Albumin Promoter,” Mol. Biol. Med., 7:173, 1990.

Tur-Kaspa, Teicher, Levine, Skoultchi and Shafritz, “Use of electroporation to introduce biologically active foreign genes into primary rat hepatocytes,” Mol. Cell Biol., 6:716-718, 1986.

Tyndall, La Mantia, Thacker, Favaloro, and Kamen, “A Region of the Polyoma Virus Genome Between the Replication Origin and Late Protein-Coding Sequences is Required in cis for Both Early Gene Expression and Viral DNA Replication,” Nuc. Acids. Res., 9:6231, 1981.

Vannice and Levinson, “Properties of the Human Hepatitis B Virus Enhancer: Position Effects and Cell-Type Nonspecificity,” J. Virology, 62:1305, 1988.

Varmus et al., Cell, 25:23-36, 1981.

Vasseur, Kress, Montreau, and Blangy, “Isolation and Characterization of Polyoma Virus Mutants Able to Develop in Multipotential Murine Embryonal Carcinoma Cells,” Proc Nat'l Acad. Sci. U.S.A., 77:1068, 1980.

Von Harsdorf, R., Hauck, L., Mehrhof, F., Wegenka, U., Cardoso, M. C. & Deitz, R. Cir. Res. 85, 128-136, 1999.

Wagner, Zenke, Cotten, Beug, Birnstiel, “Transferrin-polycation conjugates as carriers for DNA uptake into cells,” Proc. Nat'l Acad. Sci. USA 87(9):3410-3414, 1990.

Wang and Calame, “SV40 enhancer-binding factors are required at the establishment but not the maintenance step of enhancer-dependent transcriptional activation,” Cell, 47:241, 1986.

Wang, C-Y., Mayo, M. W., Korneluk, R. G., Goeddel, D. V., and Baldwin, A. S Jr. (1998). NF-□B Antiapoptosis: Induction of TRAF1 and TRAF2 and c-IAP1 and c-IAP2 to suppress caspase-8 activation. Science 281, 1680-1683.

Wang, Y., Su, B., Sah, V. P., Brown, J. H., Han, J. and Chien, K. R. (1998b) Cardiac hypertrophy induced by mitogen-activated protein kinase kinase 7, a specific activator for c-Jun NH2-terminal kinase in ventricular muscle cells. J. Biol. Chem., 273, 5423-6.

Weber, De Villiers, and Schaffner, “An SV40 ‘Enhancer Trap’ Incorporates Exogenous Enhancers or Generates Enhancers From its Own Sequences,” Cell, 36:983, 1984.

Weinberger, Jat, and Sharp, “Localization of a Repressive Sequence Contributing to B-cell Specificity in the Immunoglobulin Heavy-Chain Enhancer,” Mol. Cell. Biol., 8:988, 1984.

Weng, Czaplinski, Peltz, “Genetic and biochemical characterization of mutations in the ATPase and helicase regions of the Upfl protein,” Molecular Cellular Biol., 16:154-160, 1996.

Wilson-Rawls, Molkentin, Black, Olson, “Activated notch inhibits myogenic activity of the MADS-Box taqnscritpion factor myocyte enhancer factor 2C,” Molecular Cellular Biology, 19:2853-2862, 1999.

Winoto and Baltimore, “αβ-lineage-specific Expression of the α T-Cell Receptor Gene by Nearby Silencers,” Cell, 59:649, 1989.

WO 84/03564

WO 90/07641, 1990

Wong et al., “Appearance of b-lactamase activity in animal cells upon liposome mediated gene transfer”, Gene, 10:87-94, 1980.

Wu and Wallace, “The ligation amplification reaction (LAR)—amplification of specific DNA sequences using sequential rounds of template-dependent ligation,” Genomics, 4:560, 1989.

Wu and Wu, “Evidence for targeted gene delivery to HepG2 hepatoma cells in vitro” Biochemistry, 27:887-892, 1988.

Wu and Wu, “Receptor-mediated in vitro gene transfections by a soluble DNA carrier system”, J. Biol. Chem., 262:4429-4432, 1987.

Wu and Wu, Adv. Drug Delivery Rev., 12:159-167, 1993.

Yamauchi-Takihara, Sole, Liew, 1 ng, Liew, “Characterization of human cardiac myosin heavy chain genes,” Proc. Nat'l Acad. Sci. USA, 86(10):3504-8, 1989.

Yang, Burkholder, Roberts, Martinell and McCabe, “In vivo and in vitro gene transfer to mammalian somatic cells by particle bombardment,” Proc Nat'l Acad. Sci. USA, 87:9568-9572, 1990.

Yeung, Bockhold, Tufaro, “Efficient infection of mature skeletal muscle with herpes simplex virus vectors by using dextran sulfate as a co-receptor,” Gene Ther, 6(9):1536-44, 1999.

Yutzey, Kline, and Konieczny, “An Internal Regulatory Element Controls Troponin I Gene Expression,” Mol. Cell. Biol., 9:1397, 1989.

Zelenin et al., “High-velocity mechanical DNA transfer of the chloramphenicol acetyltransferase gene into rodent liver, kidney and mammary gland cells in organ explants and in vivo”, FEBS Lett., 280:94-96, 1991.

Zhang, Wang, Montalvo, “Smubp-2 represses the Epstein-Bar virus lytic switch promoter,” Virology, 255:160-170, 1999.

Ziober and Kramer, “Identification and characterization of the cell type-specific and developmentally regulated α7 integrin gene promoter,” J. Bio. Chem., 271(37):22915-22, 1996.

What is claimed is:

1. A peptide comprising at least 6 consecutive amino acids of SEQ ID NO:2 or 3.

2. The peptide of claim 1, comprising at least 8 consecutive amino acids of SEQ ID NO:2 or 3.

3. The peptide of claim 1, comprising at least 10 consecutive amino acids of SEQ ID NO:2 or 3.

4. The peptide of claim 1, comprising at least 15 consecutive amino acids of SEQ ID NO:2 or 3.

5. The peptide of claim 1, comprising at least 25 consecutive amino acids of SEQ ID NO:2 or 3.

6. The peptide of claim 1, comprising at least 50 consecutive amino acids of SEQ ID NO:2 or 3.

7. The peptide of claim 1, wherein said peptide comprises the sequence of SEQ ID NO:2 or 3.

8. The peptide of claim 1, wherein said peptide is fused to a heterologous amino acid sequence.

9. The peptide of claim 8, wherein the heterologous amino acid sequence encode a selectable or screenable marker.

10. The peptide of claim 8, wherein the heterologous amino acid sequences comprises a nuclear localization signal.

11. An expression construct comprising a nucleic acid segment encoding at least 5 consecutive amino acids of SEQ ID NO:2 or 3, wherein the nucleic acid sequence is under the transcriptional control of a promoter operable in eukaryotic cells.

12. The expression construct of claim 1 1, wherein the nucleic acid sequence encodes SEQ ID NO:2 or 3.

13. The expression construct of claim 11, wherein the promoter is a tissue specific promoter, a constitutive promoter or an inducible promoter.

14. The expression construct of claim 13, wherein the promoter is a tissue specific promoter active in cardiac cells or neuronal cells.

15. The expression construct of claim 11, wherein the expression construct comprises a non-viral vector.

16. The expression construct of claim 11, wherein the non-viral vector is entrapped in a liposome.

17. The expression construct of claim 11, wherein the expression construct comprises a viral vector.

18. The expression construct of claim 17, wherein the viral vector is an adenoviral vector, an adeno-associated viral vector, a retroviral vector, a vaccinia viral vector, a herpesviral vector or a polyoma viral vector.

19. The expression construct of claim 11, wherein the nucleic acid segment is positioned sense to the promoter.

20. The expression construct of claim 11, wherein the nucleic acid segment is positioned antisense to the promoter.

21. An oligonucleotide consisting essentially of 12 to 200 base pairs, wherein the nucleic acid sequence comprises at least 10 consecutive bases of SEQ ID NO:1.

22. The oligonucleotide of claim 21, wherein the nucleic acid segment comprises at least 11, 12, 13, 14, 15, 16, 17, 18, 19 or 20 consecutive bases of SEQ ID NO:1.

23. The oligonucleotide of claim 21, wherein the nucleic acid segment comprises at least 30, 40, 50, 60, 70, 80, 90, or 100 consecutive nucleotides of SEQ ID NO:1.

24. The oligonucleotide of claim 21, wherein the nucleic acid segment comprises 110, 120, 130, 140, 150, 160, 170, 180, 190 or 200 consecutive nucleotides of SEQ ID NO:1.

25. The oligonucleotide of claim 21, wherein the nucleic acid segment comprises SEQ ID NO:1.

26. A method of inhibiting the proliferation of a cell comprising administering to the cell an effective amount of a composition that increases the level and/or activity of a HOP protein in said cell.

27. The method of claim 26, wherein the cell is a cardiac cell, a lung cell, a brain cell, a neuronal cell, or a liver cell.

28. The method of claim 26, wherein the cell is located in a subject.

29. The method of claim 26, wherein the composition comprises a HOP protein.

30. The method of claim 29, wherein the HOP protein is comprised within a liposome.

31. The method of claim 26, wherein the composition comprises an expression construct encoding a HOP protein.

32. The method of claim 31, wherein the expression construct is comprised within a viral particle.

33. The expression construct of claim 31, wherein the expression vector is an adenoviral vector, an adeno-associated viral vector, a retroviral vector, a vaccinia viral vector, a herpesviral vector or a polyoma viral vector.

34. The method of claim 31, wherein the expression construct is comprised within a liposome.

35. The method of claim 26, wherein the composition is a small molecule.

36. A method of promoting the proliferation of a cell comprising administering to the cell an effective amount of a composition that reduces the levels and/or activity of a HOP protein.

37. The method of claim 36, wherein the cell is a cardiac cell, a lung cell, a brain cell, a neuronal cell, or a liver cell.

38. The method of claim 36, wherein the cell is located in a subject.

39. The method of claim 36, wherein the composition comprises an anti-HOP antibody.

40. The method of claim 39, wherein the anti-HOP antibody is comprised within a liposome.

41. The method of claim 36, wherein the composition comprises an expression construct encoding a single chain anti-HOP antibody, a HOP ribozyme, a ds HOP RNA or a HOP antisense molecule.

42. The method of claim 41, wherein the expression construct is comprised within a viral particle.

43. The expression construct of claim 41, wherein the expression vector is an adenoviral vector, an adeno-associated viral vector, a retroviral vector, a vaccinia viral vector, a herpesviral vector or a polyoma viral vector.

44. The method of claim 41, wherein the expression construct is comprised within a liposome.

45. The method of claim 36, wherein the composition is a small molecule.

46. A method of producing a HOP protein comprising:
(a) providing a cell comprising an expression construct comprising a nucleic acid segment encoding the sequence of SEQ ID NO:2 or 3, wherein the nucleic acid sequence is under the transcriptional control of a promoter operable in the cell; and
(b) culturing the cell under conditions whereby the HOP protein is produced.

47. A cell comprising a nucleic acid segment sequence encoding the sequence of SEQ ID NO:2 or 3, wherein the nucleic acid segment is under the transcriptional control of a promoter, other than the native HOP promoter, that is operable in the cell.

48. The cell of claim 47, wherein the cell is a cardiac cell, a lung cell, a brain cell, a neuronal cell, or a liver cell.

49. The cell of claim 47, wherein the promoter is a tissue specific promoter, a constitutive promoter or an inducible promoter.

50. The cell of claim 49, wherein the promoter is a tissue specific promoter active in cardiac cells or neuronal cells.

51. A method of identifying a novel cardiac transcription factor comprising:
(a) providing a HOP protein;
(b) contacting the HOP protein with a cellular extract;
(c) determining the binding of the HOP protein to a molecule in the cellular extract; and
(d) identifying the molecule bound to the HOP protein.

52. A method of diagnosing congenital heart disease in a subject comprising:
(a) obtaining a protein-containing sample from a subject; and
(b) assessing the level of HOP protein in the sample;
wherein a reduced level of HOP protein is indicative of congenital heart disease.

53. A method of diagnosing congenital heart disease in a subject comprising:
(a) obtaining a protein-containing sample from a subject; and
(b) assessing the structure of HOP protein in the sample;
wherein an alteration in the structure of HOP protein is indicative of congenital heart disease.

54. A method of diagnosing congenital heart disease in a subject comprising:
(a) obtaining an mRNA-containing sample from a subject; and
(b) assessing the level of HOP mRNA in the sample;
wherein a reduced level of HOP mRNA is indicative of congenital heart disease.

55. A method of diagnosing congenital heart disease in a subject comprising:
(a) obtaining a nucleic acid-containing sample from a subject; and
(b) identifying a sequence mutation of HOP nucleic acid in the sample;
wherein a mutation in HOP nucleic acid is indicative of congenital heart disease.

56. A method of promoting cardiac myocyte differentiation comprising contacting an undifferentiated cardiac myocyte with a composition that increases the expression and/or activity of HOP protein in the cardiac myocyte.

57. A method of inhibiting cardiac myocyte differentiation comprising contacting an undifferentiated cardiac myocyte with a composition that reduces the expression and/or activity of HOP protein in the cardiac myocyte.

58. A method of reversing cardiac myocyte differentiation comprising contacting a differentiated cardiac myocyte with a composition that reduces the expression and/or activity of HOP protein in the cardiac myocyte.

59. A non-human transgenic animal, cells of which lack at least one functional native HOP allele.

60. The transgenic animal of claim 59, wherein the cells of the transgenic animal lacks both functional native HOP alleles.

61. The transgenic animal of claim 60, wherein the cells of the transgenic animal further comprises a HOP transgene that is under the control of regulatable promoter.

62. A method of treating cardiac hypertrophy in a subject comprising administering to the subject an effective amount of a composition that increases the level and/or activity of a HOP protein in said cell.

63. A method of shutting off a fetal gene program in a cardiac myocyte comprising contacting the cardiac myocyte with an effective amount of a composition that increases the level and/or activity of a HOP protein in said cell.

64. A method of inducing neuronal tissue growth in a subject comprising administering to the subject an effective amount of a composition that reduces the levels and/or activity of a HOP protein.

65. The method of claim 64, wherein said subject has suffered a neuronal tissue injury.

66. An isolated and purified polynucleotide encoding the sequence of SEQ ID NO:2 or 3.

67. The isolated and purified polynucleotide of claim 66, wherein the sequence is SEQ ID NO:1.

68. The isolated and purifed polynucleotide encoding a fragment of SEQ ID NO:2 or 3.
